(19) United States
(12) Patent Application Publication
(10) Pub. No.: US 2022/0296572 A1
(43) Pub. Date: Sep. 22, 2022

(54) IMMUNOSUPPRESSIVE THERAPIES FOR USE WITH CARDIOMYOCYTE CELL THERAPIES, AND ASSOCIATED METHODS AND COMPOSITIONS

(71) Applicant: Sana Biotechnology, Inc., Seattle, WA (US)

(72) Inventors: Robert Scott THIES, Seattle, WA (US); Charles E. MURRY, Seattle, WA (US)

(73) Assignee: Sana Biotechnology, Inc., Seattle, WA (US)

(21) Appl. No.: 17/685,180

(22) Filed: Mar. 2, 2022

Related U.S. Application Data
(60) Provisional application No. 63/155,946, filed on Mar. 3, 2021.

(51) Int. Cl. A61K 31/436; A61K 35/34; A61K 31/343; A61P 37/06

(52) U.S. Cl. CPC A61K 31/436; A61K 35/34; A61K 31/343; A61P 37/06

(57) ABSTRACT

Provided are methods and uses related to cardiomyocyte cell therapies involving the administration of immunosuppressive agents for reducing or preventing rejection of administered allogeneic cardiomyocytes. In some embodiments, the methods are for treating a subject with a heart disease or condition in combination with a cardiomyocyte cell therapy.

### CROSS REFERENCE TO RELATED APPLICATION

This application claims priority to U.S. Provisional Application No. 63/155,946, filed Mar. 3, 2021, entitled “IMMUNOSUPPRESSIVE THERAPIES FOR USE WITH CARDIOMYOCYTE CELL THERAPIES, AND ASSOCIATED METHODS AND COMPOSITIONS,” the contents of which are incorporated by reference in their entirety for all purposes.

### FIELD

The present disclosure relates in some aspects to methods and uses related to cardiomyocyte cell therapies involving the administration of an immunosuppressive therapy for attenuating or preventing rejection of administered allogeneic cardiomyocytes. In some embodiments, the methods are for treating a subject with a heart condition or disease in combination with a cardiomyocyte cell therapy.

### BACKGROUND

Various strategies are available for treating heart conditions and diseases. Allogeneic primary cardiomyocytes, as well as differentiated cardiomyocytes derived from allogeneic pluripotent cells, have been investigated for use in treating heart conditions and diseases, including myocardial infarction (MI). The propensity for the recipient's immune system to reject allogeneic cells, however, reduces the potential efficacy of such treatments. While immunosuppressive agents have been investigated for use in attenuating and preventing rejection, they often have unwanted side effects, do not sufficiently prolong survival of the allogeneic cells or tissues in the recipient, or both. Thus, there remains a need for improved methods of immunosuppression, including for use with cardiomyocyte cell therapies. Provided herein are embodiments that meet such needs.

### SUMMARY

Provided herein is a method of attenuating or preventing rejection of a cardiomyocyte cell therapy in a subject, the method comprising administering to a subject (i) tacrolimus and (ii) mycophenolic acid (MPA), a pharmaceutical salt thereof, and/or a proform thereof prior to, concurrent with, and/or subsequent to administration of a cardiomyocyte cell therapy to the subject.

Also provided here is a method of attenuating or preventing rejection of a cardiomyocyte cell therapy in a subject, the method comprising administering to a subject a cardiomyocyte cell therapy prior to, concurrent with, and/or subsequent to administration of (i) tacrolimus and (ii) mycophenolic acid (MPA), a pharmaceutical salt thereof, and/or a proform thereof to the subject.

Also provided herein is a method of attenuating or preventing rejection of a cardiomyocyte cell therapy in a subject, the method comprising: (1) administering a cardiomyocyte cell therapy to a subject; and (2) administering (i) tacrolimus and (ii) mycophenolic acid (MPA), a pharmaceutical salt thereof, and/or a proform thereof to the subject, wherein administration of (i) tacrolimus and (ii) MPA, a pharmaceutical salt thereof, and/or a proform thereof is prior to, concurrent with, and/or subsequent to administration of the cardiomyocyte cell therapy to the subject.

Also provided herein is use of (i) tacrolimus and (ii) mycophenolic acid (MPA), a pharmaceutical salt thereof, and/or a proform thereof for attenuating or preventing rejection of a cardiomyocyte cell therapy in a subject who has received, is receiving, and/or is to receive a cardiomyocyte cell therapy.

Also provided herein is use of a cardiomyocyte cell therapy for attenuating or preventing rejection of the cardiomyocyte cell therapy in a subject who has received, is receiving, and/or is to receive (i) tacrolimus and (ii) mycophenolic acid (MPA), a pharmaceutical salt thereof, and/or a proform thereof.

Also provided herein is use of a cardiomyocyte cell therapy; and (i) tacrolimus and (ii) mycophenolic acid (MPA), a pharmaceutical salt thereof, and/or a proform thereof for attenuating or preventing rejection of the cardiomyocyte cell therapy in a subject, wherein the subject is administered (i) tacrolimus and (ii) MPA, a pharmaceutical salt thereof, and/or a proform thereof prior to, concurrent with, and/or subsequent to the subject being administered the cardiomyocyte cell therapy.

Also provided herein is use of (i) tacrolimus and (ii) mycophenolic acid (MPA), a pharmaceutical salt thereof, and/or a proform thereof in the manufacture of a medicament for attenuating or preventing rejection of a cardiomyocyte cell therapy in a subject who has received, is receiving, and/or is to receive a cardiomyocyte cell therapy.

In some embodiments, mycophenolic acid (MPA), a pharmaceutical salt thereof, and/or a proform thereof is MPA. In some embodiments, mycophenolic acid (MPA), a pharmaceutical salt thereof, and/or a proform thereof is mycophenolate mofetil (MMF). In some embodiments, mycophenolic acid (MPA), a pharmaceutical salt thereof, and/or a proform thereof is mycophenolate sodium (MS). In some embodiments, mycophenolic acid (MPA), a pharmaceutical salt thereof, and/or a proform thereof is MPA and MMF. In some embodiments, mycophenolic acid (MPA), a pharmaceutical salt thereof, and/or a proform thereof is MPA and MS. In some embodiments, mycophenolic acid (MPA), a pharmaceutical salt thereof, and/or a proform thereof is MMF and MS. In some embodiments, mycophenolic acid (MPA), a pharmaceutical salt thereof, and/or a proform thereof is MPA, MMF, and MS.

In some embodiments, the cardiomyocyte cell therapy is a pharmaceutical composition comprising cardiomyocytes and a pharmaceutically acceptable carrier. In some embodiments, the cardiomyocyte cell therapy is administered as a suspension of cardiomyocytes or as an engineered tissue graft comprising cardiomyocytes and a matrix. In some embodiments, the cardiomyocyte cell therapy is administered as a suspension of cardiomyocytes. In some embodiments, the cardiomyocyte cell therapy is administered as an engineered tissue graft comprising cardiomyocytes and a matrix. In some embodiments, the cardiomyocyte cell therapy is administered as a suspension of cardiomyocytes.

In some embodiments, administering tacrolimus includes administering at least one dose of tacrolimus prior to, concurrent with, and/or subsequent to administration of the cardiomyocyte cell therapy to the subject.

In some embodiments, at least one dose of tacrolimus is administered to the subject prior to administration of the cardiomyocyte cell therapy to the subject. In some embodiments, the at least one dose of tacrolimus is administered about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to administration of the cardiomyocyte cell therapy to the subject. In some embodiments, a first dose of tacrolimus is administered to the subject about 7 days prior to administration of the cardiomyocyte cell therapy to the subject. In some embodiments, a first dose of tacrolimus is administered to the subject about 6 days prior to administration of the cardiomyocyte cell therapy to the subject. In some embodiments, a first dose of tacrolimus is administered to the subject about 5 days prior to administration of the cardiomyocyte cell therapy to the subject. In some embodiments, a first dose of tacrolimus is administered to the subject about 4 days prior to administration of the cardiomyocyte cell therapy to the subject. In some embodiments, a first dose of tacrolimus is administered to the subject about 3 days prior to administration of the cardiomyocyte cell therapy to the subject. In some embodiments, a first dose of tacrolimus is administered to the subject about 2 days prior to administration of the cardiomyocyte cell therapy to the subject. In some embodiments, a first dose of tacrolimus is administered to the subject about 1 day prior to administration of the cardiomyocyte cell therapy to the subject.

In some embodiments, administering tacrolimus includes administering at least one dose of tacrolimus concurrent with administration of the cardiomyocyte cell therapy to the subject. In some embodiments, a first dose of tacrolimus is administered to the subject concurrent with administration of the cardiomyocyte cell therapy to the subject.

In some embodiments, administering tacrolimus includes administering at least one dose of tacrolimus subsequent to administration of the cardiomyocyte cell therapy to the subject.

In some embodiments, the at least one dose of tacrolimus is administered to the subject about 1 hour subsequent to, about 2 hours subsequent to, about 4 hours subsequent to, about 6 hours subsequent to, about 8 hours subsequent to, about 10 hours subsequent to, about 12 hours, or about 24 hours subsequent to administration of the cardiomyocyte cell therapy to the subject.

In some embodiments, a first dose of tacrolimus is administered to the subject subsequent to administration of the cardiomyocyte cell therapy to the subject. In some embodiments, a first dose of tacrolimus is administered to the subject about 1 hour subsequent to, about 2 hours subsequent to, about 4 hours subsequent to, about 6 hours subsequent to, about 8 hours subsequent to, about 10 hours subsequent to, about 12 hours, or about 24 hours subsequent to administration of the cardiomyocyte cell therapy to the subject.

In some embodiments, the total daily dosage of tacrolimus administered to the subject yields a blood trough level of between about 1 ng/mL and about 30 ng/mL, between about 2 ng/mL and about 25 ng/mL, between about 5 ng/mL and about 20 ng/mL, or between about 10 ng/mL and about 15 ng/mL, inclusive of each. In some embodiments, the total daily dosage of tacrolimus administered to the subject yields a blood trough level of between about 5 ng/mL and about 10 ng/mL, inclusive of each. In some embodiments, the total daily dosage of tacrolimus administered to the subject yields a blood trough level of between about 10 ng/mL and about 15 ng/mL, inclusive of each. In some embodiments, the total daily dosage of tacrolimus administered to the subject yields a blood trough level of about 5 ng/mL. In some embodiments, the total daily dosage of tacrolimus administered to the subject yields a blood trough level of about 10 ng/mL. In some embodiments, the total daily dosage of tacrolimus administered to the subject yields a blood trough level of about 15 ng/mL.

In some embodiments, tacrolimus is administered at least once daily. In some embodiments, the total daily dosage of tacrolimus is provided as a single dose per day. In some embodiments, the total daily dosage of tacrolimus is divided between 2 doses, 3 doses, or 4 doses per day. In some embodiments, the total daily dosage of tacrolimus is divided between two doses per day (bid). In some embodiments, a dose of tacrolimus is administered about every 12 hours. In some embodiments, the total daily dosage of tacrolimus is divided between three doses per day. In some embodiments, a dose of tacrolimus is administered about every 8 hours. In some embodiments, the total daily dosage of tacrolimus is divided between four doses per day. In some embodiments, a dose of tacrolimus is administered about every 6 hours.

In some embodiments, tacrolimus is administered daily for about 3 months, about 6 months, about 12 months, about 24 months, about 36 months, about 48 months, about 60 months, or more subsequent to administration of the cardiomyocyte cell therapy to the subject. In some embodiments, tacrolimus is administered daily for the lifetime of the cardiomyocyte cell therapy in the subject.

In some embodiments, (a) a first dose of tacrolimus is administered to the subject about 5 days prior to administration of the cardiomyocyte cell therapy to the subject; and (b) two doses of tacrolimus are administered daily to the subject (bid), beginning at the first dose of tacrolimus and continuously thereafter for the lifetime of the administered cardiomyocyte cell therapy. In some embodiments, a dose of tacrolimus is administered about every 12 hours. In some embodiments, (a) a first dose of tacrolimus is administered to the subject about 5 days prior to administration of the cardiomyocyte cell therapy to the subject; and (b) two doses of tacrolimus are administered daily to the subject (bid), wherein a dose of tacrolimus is administered every 12 hours, beginning at the first dose of tacrolimus and continuously thereafter for the lifetime of the administered cardiomyocyte cell therapy.

In some embodiments, administration of tacrolimus includes oral, subcutaneous, and/or intravenous administration. In some embodiments, administration of tacrolimus includes oral administration. In some embodiments, administration of tacrolimus includes subcutaneous administration. In some embodiments, administration of tacrolimus includes intravenous administration.

In some embodiments, (i) administration of tacrolimus includes subcutaneous administration and (ii) administration of MPA, a pharmaceutical salt thereof, and/or a proform thereof includes oral administration. In some embodiments, (i) administration of tacrolimus includes subcutaneous administration and (ii) administration of MPA, a pharmaceutical salt thereof, and/or a proform thereof includes intravenous administration.

In some embodiments, administering MPA, a pharmaceutical salt thereof, and/or a proform thereof includes administering at least one dose of MPA, a pharmaceutical salt thereof, and/or a proform thereof prior to, concurrent with, and/or subsequent to administration of the cardiomyocyte cell therapy to the subject.

In some embodiments, administering MPA, a pharmaceutical salt thereof, and/or a proform thereof includes administering at least one dose of MPA, a pharmaceutical salt thereof, and/or a proform thereof prior to administration of the cardiomyocyte cell therapy to the subject. In some embodiments, the at least one dose of MPA, a pharmaceutical salt thereof, and/or a proform thereof is administered about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to administration of the cardiomyocyte cell therapy to the subject. In some embodiments, a first dose of MPA, a pharmaceutical salt thereof, and/or a proform thereof is administered to the subject about 7 days prior to administration of the cardiomyocyte cell therapy to the subject. In some embodiments, a first dose of MPA, a pharmaceutical salt thereof, and/or a proform thereof is administered to the subject about 6 days prior to administration of the cardiomyocyte cell therapy to the subject. In some embodiments, a first dose of MPA, a pharmaceutical salt thereof, and/or a proform thereof is administered to the subject about 5 days prior to administration of the cardiomyocyte cell therapy to the subject. In some embodiments, a first dose of MPA, a pharmaceutical salt thereof, and/or a proform thereof is administered to the subject about 4 days prior to administration of the cardiomyocyte cell therapy to the subject. In some embodiments, a first dose of MPA, a pharmaceutical salt thereof, and/or a proform thereof is administered to the subject about 3 days prior to administration of the cardiomyocyte cell therapy to the subject. In some embodiments, a first dose of MPA, a pharmaceutical salt thereof, and/or a proform thereof is administered to the subject about 2 days prior to administration of the cardiomyocyte cell therapy to the subject. In some embodiments, a first dose of MPA, a pharmaceutical salt thereof, and/or a proform thereof is administered to the subject about 1 day prior to administration of the cardiomyocyte cell therapy to the subject.

In some embodiments, administering MPA, a pharmaceutical salt thereof, and/or a proform thereof includes administering at least one dose of MPA, a pharmaceutical salt thereof, and/or a proform thereof concurrent with administration of the cardiomyocyte cell therapy to the subject. In some embodiments, a first dose of MPA, a pharmaceutical salt thereof, and/or a proform thereof is administered to the subject concurrent with administration of the cardiomyocyte cell therapy to the subject.

In some embodiments, administering MPA, a pharmaceutical salt thereof, and/or a proform thereof includes administering at least one dose of MPA, a pharmaceutical salt thereof, and/or a proform thereof subsequent to administration of the cardiomyocyte cell therapy to the subject. In some embodiments, the at least one dose of MPA, a pharmaceutical salt thereof, and/or a proform thereof is administered to the subject about 1 hour subsequent to, about 2 hours subsequent to, about 4 hours subsequent to, about 6 hours subsequent to, about 8 hours subsequent to, about 10 hours subsequent to, about 12 hours, or about 24 hours subsequent to administration of the cardiomyocyte cell therapy to the subject.

In some embodiments, a first dose of MPA, a pharmaceutical salt thereof, and/or a proform thereof is administered to the subject subsequent to administration of the cardiomyocyte cell therapy to the subject, optionally about 1 hour subsequent to, about 2 hours subsequent to, about 4 hours subsequent to, about 6 hours subsequent to, about 8 hours subsequent to, about 10 hours subsequent to, about 12 hours, or about 24 hours subsequent to administration of the cardiomyocyte cell therapy to the subject.

In some embodiments, MPA, a pharmaceutical salt thereof, and/or a proform thereof is administered at least once daily. In some embodiments, the total daily dosage of MPA, a pharmaceutical salt thereof, and/or a proform thereof is provided as a single dose per day. In some embodiments, the total daily dosage of MPA, a pharmaceutical salt thereof, and/or a proform thereof is divided between two doses, three doses, or four doses per day. In some embodiments, the total daily dosage of MPA, a pharmaceutical salt thereof, and/or a proform thereof is divided between two doses per day (bid). In some embodiments, a dose of MPA, a pharmaceutical salt thereof, and/or a proform thereof is administered about every 12 hours. In some embodiments, the total daily dosage of MPA, a pharmaceutical salt thereof, and/or a proform thereof is divided between three doses per day. In some embodiments, a dose of MPA, a pharmaceutical salt thereof, and/or a proform thereof is administered about every 8 hours. In some embodiments, the total daily dosage of MPA, a pharmaceutical salt thereof, and/or a proform thereof is divided between four doses per day. In some embodiments, a dose of MPA, a pharmaceutical salt thereof, and/or a proform thereof is administered about every 6 hours.

In some embodiments, MPA, a pharmaceutical salt thereof, and/or a proform thereof is administered daily for about 3 months, about 6 months, about 12 months, about 24 months, about 36 months, about 48 months, about 60 months, or more subsequent to administration of the cardiomyocyte cell therapy to the subject. In some embodiments, MPA, a pharmaceutical salt thereof, and/or a proform thereof is administered daily for the lifetime of the administered cardiomyocyte cell therapy.

In some embodiments, (a) a first dose of MPA, a pharmaceutical salt thereof, and/or a proform thereof is administered to the subject about 5 days prior to administration of the cardiomyocyte cell therapy to the subject; and (b) two doses of MPA, a pharmaceutical salt thereof, and/or a proform thereof are administered daily to the subject (bid), beginning at the first dose and continuously thereafter for the lifetime of the administered cardiomyocyte cell therapy. In some embodiments, a dose of MPA, a pharmaceutical salt thereof, and/or a proform thereof is administered about every 12 hours. In some embodiments, (a) a first dose of MPA, a pharmaceutical salt thereof, and/or a proform thereof is administered to the subject about 5 days prior to administration of the cardiomyocyte cell therapy to the subject; and (b) two doses of MPA, a pharmaceutical salt thereof, and/or a proform thereof are administered daily to the subject (bid), wherein a dose of MPA, a pharmaceutical salt thereof, and/or a proform thereof is administered about every 12 hours, beginning at the first dose and continuously thereafter for the lifetime of the administered cardiomyocyte cell therapy.

In some embodiments, administration of MPA, a pharmaceutical salt thereof, and/or a proform thereof includes oral, subcutaneous, and/or intravenous administration. In some embodiments, administration of MPA, a pharmaceutical salt thereof, and/or a proform thereof includes oral administration. In some embodiments, administration of MPA, a pharmaceutical salt thereof, and/or a proform thereof includes subcutaneous administration. In some embodiments, administration of MPA, a pharmaceutical salt thereof, and/or a proform thereof includes intravenous administration.

In some embodiments, (i) administration of tacrolimus includes subcutaneous administration and (ii) administration of MPA, a pharmaceutical salt thereof, and/or a proform thereof includes oral administration. In some embodiments, (i) administration of tacrolimus includes subcutaneous administration and (ii) administration of MPA, a pharmaceutical salt thereof, and/or a proform thereof includes intravenous administration.

In some embodiments, a first dose of tacrolimus and a first dose of MPA, a pharmaceutical salt thereof, and/or a proform thereof are each administered to the subject on the same day. In some embodiments, tacrolimus and MPA, a pharmaceutical salt thereof, and/or a proform thereof are each administered to the subject daily. In some embodiments, the total daily dosage of tacrolimus is divided between two doses per day and the total daily dosage of MPA, a pharmaceutical salt thereof, and/or a proform thereof is divided between two doses per day.

In some embodiments, (a) a first dose of tacrolimus and a first dose of MPA, a pharmaceutical salt thereof, and/or a proform thereof are each administered to the subject about 5 days prior to administration of the cardiomyocyte cell therapy to the subject; and (b) two doses of tacrolimus and two doses of MPA, a pharmaceutical salt thereof, and/or a proform thereof are administered daily to the subject (bid), beginning at the first doses of tacrolimus and MPA, a pharmaceutical salt thereof, and/or a proform thereof and continuously thereafter for the lifetime of the administered cardiomyocyte cell therapy. In some embodiments, a dose of tacrolimus and a dose of MPA, a pharmaceutical salt thereof, and/or a proform thereof are each administered about every 12 hours. In some embodiments, (a) a first dose of tacrolimus and a first dose of MPA, a pharmaceutical salt thereof, and/or a proform thereof are each administered to the subject about 5 days prior to administration of the cardiomyocyte cell therapy to the subject; and (b) two doses of tacrolimus and two doses of MPA, a pharmaceutical salt thereof, and/or a proform thereof are administered daily to the subject (bid), wherein a dose of tacrolimus and a dose of MPA, a pharmaceutical salt thereof, and/or a proform thereof are each administered about every 12 hours, beginning at the first doses of tacrolimus and MPA, a pharmaceutical salt thereof, and/or a proform thereof and continuously thereafter for the lifetime of the administered cardiomyocyte cell therapy.

In some embodiments, a pharmaceutical salt thereof, and/or a proform thereof is mycophenolate mofetil (MMF). In some embodiments, administration of MMF includes oral administration. In some embodiments, administration of MMF includes subcutaneous administration. In some embodiments, administration of MMF includes intravenous administration.

In some embodiments, (i) administration of tacrolimus includes subcutaneous administration and (ii) administration of MMF includes oral administration. In some embodiments, (i) administration of tacrolimus includes subcutaneous administration and (ii) administration of MMF includes intravenous administration.

In some embodiments, the total daily dosage of MMF is between about 500 mg and about 3000 mg, between about 1000 mg and about 2500 mg, or between about 1500 mg and about 2000 mg, inclusive of each. In some embodiments, the total daily dosage of MMF is about 1000 mg, about 1500 mg, about 2000 mg, or about 2500 mg.

In some embodiments, MPA, a pharmaceutical salt thereof, and/or a proform thereof is mycophenolate sodium (MS). In some embodiments, the total daily dosage of MS is between about 360 mg and about 2700 mg, between about 720 mg and about 2160 mg, or between about 720 mg and about 1620 mg, inclusive of each. In some embodiments, the total daily dosage of MS is about 360 mg, about 720 mg, about 1080 mg, or about 1440 mg.

In some embodiments, the cardiomyocytes are allogeneic to the subject. In some embodiments, the cardiomyocytes are primary cardiomyocytes. In some embodiments, the cardiomyocytes are derived from pluripotent stem cells (PSCs). In some embodiments, the PSCs are induced PSCs (iPSCs).

In some embodiments, administration of the cardiomyocyte cell therapy includes delivery into a subject's heart tissue, optionally by intravenous injection, intraarterial injection, intracoronary injection, intramuscular injection, intraperitoneal injection, intramyocardial injection, trans-endocardial injection, trans-epicardial injection, and/or infusion.

In some embodiments, the cardiomyocyte cell therapy contains between about 5×10⁸ and 1×10¹⁰ cardiomyocytes, inclusive of each. In some embodiments, the cardiomyocyte cell therapy contains between about 1×10⁹ and about 5×10⁹ cardiomyocytes, inclusive of each. In some embodiments, the cardiomyocyte cell therapy contains a pharmaceutically acceptable carrier.

In some embodiments, the subject has a heart disease or condition. In some embodiments, the heart disease or condition is pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, other cardiomyopathy, myocarditis, myocardial infarction, myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, or cardiovascular disease. In some embodiments, the heart disease or condition is myocardial infarction (MI).

In some embodiments, the subject is a candidate for a Left Ventricular Assist Device (LVAD). In some embodiments, the subject has a LVAD at the time of administration of the cardiomyocyte cell therapy and/or at the time of administration of tacrolimus and MPA, a pharmaceutical salt thereof, and/or a proform thereof. In some embodiments, the subject has a LVAD at the time of administration of the cardiomyocyte cell therapy. In some embodiments, the subject has a LVAD at the time of administration of tacrolimus. In some embodiments, the subject has a LVAD at the time of administration of MPA, a pharmaceutical salt thereof, and/or a proform thereof. In some embodiments, the subject has a LVAD at the time of administration of tacrolimus and MPA, a pharmaceutical salt thereof, and/or a proform thereof. In some embodiments, the subject is a human. In some embodiments, the cardiomyocytes are human.

### BRIEF DESCRIPTION OF THE DRAWINGS

FIG. 1A shows engrafted allogeneic cardiomyocytes differentiated from rhesus induced pluripotent stem cells (riPSC-CMs) 16 weeks after delivery of the cells into the infarcted heart of a rhesus recipient, as shown by staining for slow skeletal troponin I (ssTnI).

FIG. 1B shows T cells at the site of the engraftment 16 weeks after delivery of allogeneic cardiomyocytes, as indicated by staining for CD3.

### DETAILED DESCRIPTION

Provided herein are methods of attenuating or preventing rejection of a cardiomyocyte cell therapy in a subject administered a cardiomyocyte cell therapy, involving administration of an immunosuppressive therapy of tacrolimus and mycophenolic acid (MPA), a pharmaceutical salt thereof (e.g. mycophenolate sodium; MS), and/or a proform thereof (e.g. mycophenolate mofetil; MMF)). In some embodiments, the subject has a heart disease or condition, such as myocardial infarction (MI). Thus, in some embodiments, the cardiomyocyte cell therapy is for the treatment of the MI. In some embodiments, the cardiomyocyte cell therapy is delivered to the site of the MI, or within a particular proximity of the site of the MI. In some embodiments, the cardiomyocyte cell therapy is delivered to the site of the MI.

In some embodiments, the cardiomyocyte cell therapy is a pharmaceutical composition comprising cardiomyocytes and a pharmaceutically acceptable carrier. In some embodiments, the cardiomyocyte therapy is administered as a suspension of cardiomyocytes or as an engineered tissue graft comprising cardiomyocytes and a matrix. In some embodiments, the cardiomyocytes are allogeneic to the subject. In some embodiments, the cardiomyocytes are primary cardiomyocytes or are derived from pluripotent stem cells (PSCs), such as induced pluripotent stem cells (iPSCs). In any of the provided embodiments, the administration of tacrolimus and mycophenolic acid (MPA), a pharmaceutical salt thereof (e.g. MS), and/or a proform thereof (e.g. MMF) attenuates or prevents rejection of the administered cardiomyocyte cell therapy, i.e. the persistence and/or viability of the delivered cardiomyocytes is increased as compared to the absence of tacrolimus and mycophenolic acid (MPA), a pharmaceutical salt thereof (e.g. MS), and/or a proform thereof (e.g. MMF), or as compared to administration of a different immunosuppressive therapy.

Also provided are combinations and articles of manufacture, such as kits, that contain a composition comprising cardiomyocytes, tacrolimus, and/or mycophenolic acid (MPA), a pharmaceutical salt thereof (e.g. MS), and/or a proform thereof (e.g. MMF), and uses of such compositions and combinations to attenuate or prevent rejection of a cardiomyocyte cell therapy.

Cell, tissue, and organ transplants, such as allogeneic cell-based therapies, are under investigation for their ability to treatment diseases and disorders, including heart diseases and conditions. Because organs and tissues for transplant are often in short supply, the ability to generate and/or administer cells or an engineered tissue graft can provide improved access to patients in need thereof.

Allogeneic cells are typically immunogenic to a subject receiving the cells, which can result in limited or no persistence of the administered cells (i.e. rejection of the cardiomyocyte cell therapy), thereby reducing the efficacy of the cells. Thus, methods for preventing or reducing rejection of allogeneic cells by recipient immune systems have been investigated, and include engineering the cells to be hypoimmunogenic and/or providing immunosuppressive therapy to recipients. Strategies for engineering cells to be hypoimmunogenic are known, and include but are not limited to methods as described in WO 2020/018615, WO 2020/231882, WO 2018/132783; Han et al., PNAS (2019) 116(21):10441-46; and Zhao et al., iScience (2020) 23(6):101162.

Alternatively, allogeneic cells can be provided with an immunosuppressive therapy to reduce or prevent rejection of the transplanted cells. A number of immunosuppressive therapies have been studied for their use in organ transplant rejection, including tacrolimus (also known as Prograf®; formerly known as FK506) and cyclosporine A (CsA).

In certain contexts, however, available approaches to attenuating or preventing rejection of allogeneic cells, such as those comprised by a cardiomyocyte cell therapy, may not always be entirely satisfactory. In some cases, the available approaches do not allow administered cells to sufficiently engraft, persist, and/or proliferate in vivo, or are only effective at preventing rejection of administered cells for a limited amount of time Immunosuppressive regimens also often cause unwanted side effects, including cutaneous and gastrointestinal effects.

In some aspects, the provided methods and uses achieve attenuation or prevention of rejection of a cardiomyocyte cell therapy as compared to alternative methods, such as alternative methods involving only the administration of the cardiomyocyte cell therapy but not in combination with tacrolimus and mycophenolic acid (MPA), a pharmaceutical salt thereof (e.g. MS), and/or a proform thereof (e.g. MMF). In some embodiments, the methods are advantageous by virtue of initiating administration of tacrolimus and mycophenolic acid (MPA), a pharmaceutical salt thereof (e.g. MS), and/or a proform thereof (e.g. MMF) prior to (e.g. between about 3-7 days before, such as 5 days before) or concurrently with administration of a cardiomyocyte cell therapy, thereby reducing the likelihood that administered cardiomyocytes in the subject will be rejected. In particular, observations herein demonstrate that a combination of tacrolimus and mycophenolic acid (MPA), a pharmaceutical salt thereof (e.g. MS), and/or a proform thereof (e.g. MMF) allows a cardiomyocyte cell therapy composition to persist for a substantial period of time following administration of the cell therapy.

The provided methods are also based on observations that certain immunosuppressive therapies may cause unwanted side effects. In some aspects, the provided methods and uses have reduced side effects as compared to other immunosuppressive therapies. In some aspects, the provided methods and uses do not cause or do not cause substantial unwanted side effects. In some aspects, the provided methods and uses are associated with fewer side effects as compared to other immunosuppressive therapies by virtue of causing less suppression of a subject's immune system.

The provided methods and uses are also advantageous by virtue of being amenable to subjects with a left ventricular assist device (LVAD). In particular, some subjects who are candidates for the cardiomyocyte cell therapies described herein may have a LVAD. The combination of tacrolimus and MMF is used to treat subjects with LVAD, such that the immunosuppression regimen of a subject with a LVAD who is then administered a cardiomyocyte cell therapy does not have to be modified or augmented. Thus, the provided methods are uses are suitable for a subject who has a LVAD and is then administered the cardiomyocyte cell therapy.

Tacrolimus is a macrolide antibiotic and calcineurin inhibitor used to prevent organ transplant in subjects receiving allogeneic kidney, liver, or heart transplant. Tacrolimus binds to FK-binding proteins, thereby inhibiting calcineurin, a calcium- and calmodulin-dependent phosphatase. Inhibition of calcineurin inhibits translocation of the NFAT family of transcription factors, reducing transcriptional activation of genes such as interleukin-2 (IL-2), tumor necrosis factor-alpha (TNFa), IL-3, IL-4, CD40L, granulocyte-macrophage colony-stimulating factor (GMCSF), and interferon-gamma (IFNg). Ultimately, the development, homeostasis, survival, and function of T cells is reduced, lowering immune responses in subjects. Tacrolimus exhibits similar effects to CsA, but at concentrations 100 times lower. Thomson and Zeevi, Ther. Drug. Monit. (1995) 17(6):584-91. Barbarino et al., Pharmacogenet. Genomics (2013) 23(10):563-85.

Mycophenolate mofetil (MMF; also known as CellCept®) is a prodrug of mycophenolic acid (MPA), and is approved for prophylaxis of organ rejection in recipients of allogeneic kidney, heart, or liver transplants. MMF is an inhibitor of inosine monophosphate dehydrogenase (IMPDH). IMPDH is the rate-limiting enzyme in de novo synthesis of guanosine nucleotides. As T- and B-lymphocytes are more dependent on this pathway than other cell types, MMF differentially affects T- and B-lymphocytes, thereby exerting immunosuppressive effects. MPA, as well as MMF, can also induce apoptosis of activated T-lymphocytes, eliminating clones of cells responding to antigenic stimulation. Further, by depleting guanosine nucleotides, MPA, as well as MMF, suppress glycosylation and expression of some adhesion molecules, thus decreasing recruitment of lymphocytes and monocytes to site of inflammation and graft rejection. MPA, as well as MMF, also contribute to suppression of the inducible form of nitric oxide synthase (iNOS), reducing tissue damage. Allison and Eugui, Immunopharm. (2000) 47(2-3):85-118.

Mycophenolate sodium (MS; also known as Myfortic®) is an enteric coated form of MPA. MS is approved for prophylaxis of organ transplant in recipients of allogeneic lung and kidney transplants. MS is also used off-label for prophylaxis of organ transplant in recipients of liver and heart transplant. MS is considered to be therapeutically equivalent to MMF in transplant recipients and has a comparable safety profile. Kobashigawa et al., J. Heart Lung Transplant. (2006) 25(8):935-41; and Salvadori et al., Amer. J. Transplant. (2004) 4(2):231-36. In particular, 720 mg of MS is considered to be therapeutically equivalent to 1,000 mg of MMF.

Among the provided embodiments, the methods involve administration of an immunosuppressive therapy of tacrolimus and MPA, a pharmaceutical salt thereof (e.g. MS), and/or a proform thereof (e.g. MMF) in combination with administration of a cardiomyocyte cell therapy. In some aspects, tacrolimus and/or MPA, a pharmaceutical salt thereof (e.g. MS), and/or a proform thereof (e.g. MMF) reduce the immune response of a recipient to administered allogeneic cardiomyocytes.

In some embodiments, the provided embodiments involve initiating administration of tacrolimus and/or MPA, a pharmaceutical salt thereof (e.g. MS) and/or a proform thereof (e.g. MMF), prior to, concurrent with, and/or subsequent to administration of the cardiomyocyte cell therapy. In particular embodiments, administration of tacrolimus and/or MPA, a pharmaceutical salt thereof (e.g. MS) and/or a proform thereof (e.g. MMF) is initiated between at or about 7 days prior to and at or about 1 day after administration of the cardiomyocyte cell therapy. In particular embodiments, administration of tacrolimus and/or MPA, a pharmaceutical salt thereof (e.g. MS) and/or a proform thereof (e.g. MMF) is initiated about 5 days prior to administration of the cardiomyocyte cell therapy.

In some embodiments, the provided methods involve administration of at least one dose of tacrolimus and/or at least one dose of MPA, a pharmaceutical salt thereof (e.g. MS) and/or a proform thereof (e.g. MMF) prior to administration of the cardiomyocyte cell therapy. In some embodiments, the first dose of tacrolimus and/or the first dose of MPA, a pharmaceutical salt thereof (e.g. MS) and/or a proform thereof (e.g. MMF) is administered to the subject about 7 days prior to, about 6 days prior to, about 5 days prior to, about 4 days prior to, about 3 days prior to, about 2 days prior to, about 24 hours prior to, about 12 hours prior to, about 10 hours prior to, about 8 hours prior to, about 6 hours prior to, about 4 hours prior to, about 2 hours prior to, or about 1 hour prior to administration of the cardiomyocyte cell therapy to the subject. In some embodiments, the first dose of tacrolimus and/or the first dose of MPA, a pharmaceutical salt thereof (e.g. MS) and/or a proform thereof (e.g. MMF) is administered to the subject about 6 days prior to, about 5 days prior to, or about 4 days prior to administration of the cardiomyocyte cell therapy to the subject. In some embodiments, the first dose of tacrolimus and/or the first dose of MPA, a pharmaceutical salt thereof (e.g. MS) and/or a proform thereof (e.g. MMF) is administered to the subject about 5 days prior to administration of the cardiomyocyte cell therapy to the subject.

In some embodiments, the first dose of tacrolimus and/or the first dose of MPA, a pharmaceutical salt thereof (e.g. MS) and/or a proform thereof (e.g. MMF) is administered to the subject about 60 minutes prior to, about 45 minutes prior to, about 30 minutes prior to, or about 15 minutes prior to administration of the cardiomyocyte cell therapy.

In some embodiments, the provided methods involve administration of at least one dose of tacrolimus and/or at least one dose of MPA, a pharmaceutical salt thereof (e.g. MS) and/or a proform thereof (e.g. MMF) concurrent with administration of the cardiomyocyte cell therapy to the subject. In some embodiments, the provided methods involve administration of a first dose of tacrolimus and/or a first dose of MPA, a pharmaceutical salt thereof (e.g. MS) and/or a proform thereof (e.g. MMF) concurrent with administration of the cardiomyocyte cell therapy to the subject.

In some embodiments, the provided methods involve administration of at least one dose of tacrolimus and/or at least one dose of MPA, a pharmaceutical salt thereof (e.g. MS) and/or a proform thereof (e.g. MMF) subsequent to administration of the cardiomyocyte cell therapy to the subject.

In some embodiments, the first dose of tacrolimus is administered to the subject about 1 hour subsequent to, about 2 hours subsequent to, about 4 hours subsequent to, about 6 hours subsequent to, about 8 hours subsequent to, about 10 hours subsequent to, about 12 hours subsequent to, or about 18 hours subsequent to administration of the cardiomyocyte cell therapy to the subject. In some embodiments, the first dose of tacrolimus is administered to the subject at least 6 hours subsequent to administration of the cardiomyocyte cell therapy to the subject. In some embodiments, the first dose of MPA, a pharmaceutical salt thereof (e.g. MS) and/or a proform thereof (e.g. MMF) is administered to the subject about 15 minutes subsequent to, about 30 minutes subsequent to, about 45 minutes subsequent to, 1 hour subsequent to, about 2 hours subsequent to, about 4 hours subsequent to, about 6 hours subsequent to, about 8 hours subsequent to, about 10 hours subsequent to, about 12 hours subsequent to, or about 18 hours subsequent to administration of the cardiomyocyte cell therapy to the subject. In some embodiments, the first dose of MPA, a pharmaceutical salt thereof (e.g. MS) and/or a proform thereof (e.g. MMF) is administered to the subject within about 1 hour subsequent to administration of the cardiomyocyte cell therapy to the subject.

In some embodiments, the provided methods involve administering tacrolimus and/or MPA, a pharmaceutical salt thereof (e.g. MS), and/or a proform thereof (e.g. MMF) prior to and concurrent with administration of the cardiomyocyte cell therapy to the subject. In some embodiments, the provided methods involve administering tacrolimus and/or MPA, a pharmaceutical salt thereof (e.g. MS), and/or a proform thereof (e.g. MMF) prior to and subsequent to administration of the cardiomyocyte cell therapy to the subject. In some embodiments, the provided methods involve administering tacrolimus and/or MPA, a pharmaceutical salt thereof (e.g. MS), and/or a proform thereof (e.g. MMF) concurrent with and subsequent to administration of the cardiomyocyte cell therapy to the subject. In some embodiments, the provided methods involve administering tacrolimus and/or MPA, a pharmaceutical salt thereof (e.g. MS), and/or a proform thereof (e.g. MMF) prior to, concurrent with, and subsequent to administration of the cardiomyocyte cell therapy to the subject.

In some embodiments, the provided methods involve administering tacrolimus prior to and concurrent with administration of the cardiomyocyte cell therapy to the subject. In some embodiments, the provided methods involve administering tacrolimus prior to and subsequent to administration of the cardiomyocyte cell therapy to the subject. In some embodiments, the provided methods involve administering tacrolimus concurrent with and subsequent to administration of the cardiomyocyte cell therapy to the subject. In some embodiments, the provided methods involve administering tacrolimus prior to, concurrent with, and subsequent to administration of the cardiomyocyte cell therapy to the subject.

In some embodiments, the provided methods involve administering MPA, a pharmaceutical salt thereof (e.g. MS), and/or a proform thereof (e.g. MMF) prior to and concurrent with administration of the cardiomyocyte cell therapy to the subject. In some embodiments, the provided methods involve administering MPA, a pharmaceutical salt thereof (e.g. MS), and/or a proform thereof (e.g. MMF) prior to and subsequent to administration of the cardiomyocyte cell therapy to the subject. In some embodiments, the provided methods involve administering MPA, a pharmaceutical salt thereof (e.g. MS), and/or a proform thereof (e.g. MMF) concurrent with and subsequent to administration of the cardiomyocyte cell therapy to the subject. In some embodiments, the provided methods involve administering MPA, a pharmaceutical salt thereof (e.g. MS), and/or a proform thereof (e.g. MMF) prior to, concurrent with, and subsequent to administration of the cardiomyocyte cell therapy to the subject.

In some embodiments, the provided methods involve administering tacrolimus and MPA, a pharmaceutical salt thereof (e.g. MS), and/or a proform thereof (e.g. MMF) prior to and concurrent with administration of the cardiomyocyte cell therapy to the subject. In some embodiments, the provided methods involve administering tacrolimus and MPA, a pharmaceutical salt thereof (e.g. MS), and/or a proform thereof (e.g. MMF) prior to and subsequent to administration of the cardiomyocyte cell therapy to the subject. In some embodiments, the provided methods involve administering tacrolimus and MPA, a pharmaceutical salt thereof (e.g. MS), and/or a proform thereof (e.g. MMF) concurrent with and subsequent to administration of the cardiomyocyte cell therapy to the subject. In some embodiments, the provided methods involve administering tacrolimus and MPA, a pharmaceutical salt thereof (e.g. MS), and/or a proform thereof (e.g. MMF) prior to, concurrent with, and subsequent to administration of the cardiomyocyte cell therapy to the subject.

In some cases, the methods involve administration (e.g. daily administration) of the immunosuppressive therapy of tacrolimus and MPA, a pharmaceutical salt thereof (e.g. MS), and/or a proform thereof (e.g. MMF) up to or about 1 month, 2 months, 3 months, 4 months, 5 months, 6 months, 7 months, 8 months, 9 months, 10 months, 11 months, 12 months, 24 months, 36 months, 48 months, 60 months, or more after the subject as received administration of the cardiomyocyte cell therapy. In some embodiments, the methods involve administration (e.g. daily administration) of the immunosuppressive therapy of tacrolimus and MPA, a pharmaceutical salt thereof (e.g. MS), and/or a proform thereof (e.g. MMF) throughout the lifespan of the cardiomyocyte cell therapy in the subject.

The section headings used herein are for organizational purposes only and are not to be construed as limiting the subject matter described.

### I. METHODS AND USES FOR IMMUNOSUPPRESSIVE THERAPY IN COMBINATION WITH A CARDIOMYOCYTE CELL THERAPY

Provided herein are methods of attenuating or preventing rejection of a cardiomyocyte cell therapy in a subject in need thereof that include administering to a subject (1) a cardiomyocyte cell therapy; and (2) tacrolimus and MPA, a pharmaceutical salt thereof (e.g. MS), and/or a proform thereof (e.g. MMF). In some embodiments, the subject has a heart condition or disease, such as myocardial infarction (MI), and the cardiomyocyte cell therapy is a treatment for the MI. In some embodiments, the cardiomyocyte cell therapy is a pharmaceutical composition comprising cardiomyocytes and a pharmaceutically acceptable carrier. In some embodiments, the cardiomyocyte cell therapy comprises a suspension of cardiomyocytes or an engineered tissue graft comprising cardiomyocytes. In some embodiments, the cardiomyocytes are primary cardiomyocytes. In some embodiments, the cardiomyocytes are derived from pluripotent stem cells (PSCs) (e.g. induced PSCs (iPSCs)).

Also provided are combinations and articles of manufacture, such as kits, that contain a cardiomyocyte cell therapy, tacrolimus, and/or MPA, a pharmaceutical salt thereof (e.g. MS), and/or a proform thereof (e.g. MMF), and uses of such compositions and combinations to attenuate or prevent rejection of a cardiomyocyte cell therapy administered to a subject in need thereof.

In some embodiments, methods can include administration of tacrolimus and MPA, a pharmaceutical salt thereof (e.g. MS), and/or a proform thereof (e.g. MMF) prior to, concurrent with, during, during the course of (including once and/or periodically during the course of), and/or subsequently to, the administration (e.g., initiation of administration) of the cardiomyocyte cell therapy. In some embodiments, MPA, a pharmaceutical salt thereof, and/or a proform thereof is MPA. In some embodiments, MPA, a pharmaceutical salt thereof, and/or a proform thereof is mycophenolate mofetil (MMF), a proform of MPA. In some embodiments, MPA, a pharmaceutical salt thereof and/or a proform thereof is mycophenolate sodium (MS), a pharmaceutical salt of MPA. In some embodiments, MPA, a pharmaceutical salt thereof, and/or a proform thereof is MPA and MMF. In some embodiments, MPA, a pharmaceutical salt thereof, and/or a proform thereof is MPA and MS. In some embodiments, MPA, a pharmaceutical salt thereof, and/or a proform thereof is MMF and MS. In some embodiments, MPA, a pharmaceutical salt thereof, and/or a proform thereof is MPA, MMF, and MS. In some embodiments, the subject is administered the cardiomyocyte cell therapy for treatment of a heart disease or condition (e.g. myocardial infarction; MI). Thus, in some embodiments, the subject is administered a combination of a cardiomyocyte cell therapy and an immunosuppressive therapy comprising MPA, a pharmaceutical salt thereof (e.g. MS), and/or a proform thereof (e.g. MMF) for treatment of a myocardial infarction (MI), wherein the immunosuppressive therapy is administered prior to, concurrent with, and/or subsequent to administration of the cardiomyocyte cell therapy.

In some embodiments, tacrolimus and MPA, a pharmaceutical salt thereof (e.g. MS), and/or a proform thereof (e.g. MMF) are administered to the subject as a dual agent immunosuppressive therapy. In some embodiments, tacrolimus and MPA, a pharmaceutical salt thereof (e.g. MS), and/or a proform thereof (e.g. MMF) are administered to the subject as a dual agent immunosuppressive therapy in combination with a cardiomyocyte cell therapy. In some embodiments, administration as a dual agent immunosuppressive therapy consists of a single type of treatment alone, to attenuate or prevent rejection of a cardiomyocyte cell therapy (i.e. administered cardiomyocytes), except where otherwise provided. In some embodiments, no other treatment is provided to attenuate or prevent rejection. Thus, in some embodiments, tacrolimus and MPA, a pharmaceutical salt thereof (e.g. MS), and/or a proform thereof (e.g. MMF) are provided as a dual agent immunosuppressive therapy with a cardiomyocyte cell therapy, such that no other treatment is provided to attenuate or prevent rejection beyond provision of (1) tacrolimus and MPA, a pharmaceutical salt thereof (e.g. MS), and/or a proform thereof (e.g. MMF) and (2) the cardiomyocyte cell therapy.

In some embodiments, the cardiomyocyte cell therapy is a pharmaceutical composition comprising cardiomyocytes and a pharmaceutically acceptable carrier. In some embodiments, the cardiomyocyte cell therapy comprises a suspension of cardiomyocytes. In some embodiments, the cardiomyocyte cell therapy is a suspension of cardiomyocytes, such as a suspension of dissociated cardiomyocytes. In some embodiments, the cardiomyocyte cell therapy comprises is an engineered tissue graft containing cardiomyocytes and a matrix. In some embodiments, the cardiomyocyte cell therapy does not comprise or is not an organ. In some embodiments, the cardiomyocyte cell therapy and dosage regimens for administering the cell therapy include any as described in the following subsection B under “Administration of a Cardiomyocyte Cell Therapy.”

In some embodiments, the cardiomyocytes of the cell therapy are capable of engrafting at the site of a heart disease or condition in a subject, such as at the site of a myocardial infarction (MI). In some embodiments, the cardiomyocytes are allogeneic to the subject. In some embodiments, the allogeneic cardiomyocytes are rejected and/or killed by the immune system of the subject, such as in the absence of an immunosuppressive therapy. Thus, in some embodiments, cardiomyocytes comprised by the administered cardiomyocyte cell therapy do not persist, including at the site of delivery and/or the site of a MI, because they are rejected. In some embodiments, the administration of tacrolimus and MPA, a pharmaceutical salt thereof (e.g. MS), and/or a proform thereof (e.g. MMF) attenuates or reduces the subject's immune response to the administered cardiomyocyte cell therapy. In some embodiments, administration of tacrolimus and MPA, a pharmaceutical salt thereof (e.g. MS), and/or a proform thereof (e.g. MMF) attenuates or reduces rejection of the cardiomyocyte cell therapy. Thus, in some embodiments, administration of tacrolimus and MPA, a pharmaceutical salt thereof (e.g. MS), and/or a proform thereof (e.g. MMF) allows the administered cardiomyocyte cell therapy to persist for a longer amount of time, as compared to the absence of tacrolimus and MPA, a pharmaceutical salt thereof (e.g. MS), and/or a proform thereof (e.g. MMF), or as compared to administration of a different immunosuppressive therapy.

In some embodiments, the cardiomyocyte cell therapy, tacrolimus, and MPA, a pharmaceutical salt thereof (e.g. MS), and/or a proform thereof (e.g. MMF) are provided as pharmaceutical compositions for administration to the subject. In some embodiments, the pharmaceutical compositions contain therapeutically effective amounts of one, two, or all three of the agents. In some embodiments, the pharmaceutical compositions contain subtherapeutically effective amounts of one, two or all three of the agents. In some embodiments, the agents are formulated for administration in separate pharmaceutical compositions. In some embodiments, any of the pharmaceutical compositions provided herein can be formulated in dosage forms appropriate for each route of administration.

In some embodiments, the cardiomyocyte cell therapy, as well as tacrolimus and MPA, a pharmaceutical salt thereof (e.g. MS), and/or a proform thereof (e.g. MMF) are administered to a subject or patient having a heart disease or condition, such as MI. In some aspects, the methods treat, e.g., attenuate or prevent rejection of an administered cardiomyocyte cell therapy in the subject, such as by increasing the persistence of the administered cardiomyocytes.

In some embodiments, the subject has a heart disease or condition. Exemplary heart diseases and conditions can include diseases and conditions associated with the loss (e.g. death) of cardiomyocytes, such as myocardial infarction (MI).

In some embodiments, a subject's endogenous cardiomyocytes have reduced physiological function, relative to normal cardiomyocyte physiological function (e.g. prior to the heart disease or condition). In some embodiments, a subject's endogenous cardiomyocytes have reduced physiological function as a result of a MI. In some embodiments, the physiological function of a subject's endogenous cardiomyocytes is reduced by between about 0.1% and about 50%, between about 1% and 25%, or between about 5% and 10%, inclusive of each, relative to normal cardiomyocyte physiological function. In some embodiments, the physiological function of a subject's endogenous cardiomyocytes is reduced by between about 0.1% and about 1%, inclusive of each, relative to normal cardiomyocyte physiological function. In some embodiments, the physiological function of a subject's endogenous cardiomyocytes is reduced by between about 0.5% and 5%, inclusive of each, relative to normal cardiomyocyte physiological function. In some embodiments, the physiological function of a subject's endogenous cardiomyocytes is reduced by between about 1% and 10%, inclusive of each, relative to normal cardiomyocyte physiological function. In some embodiments, the physiological function of a subject's endogenous cardiomyocytes is reduced by between about 2% and 20%, inclusive of each, relative to normal cardiomyocyte physiological function. In some embodiments, the physiological function of a subject's endogenous cardiomyocytes is reduced by between about 3% and 30%, inclusive of each, relative to normal cardiomyocyte physiological function. In some embodiments, the physiological function of a subject's endogenous cardiomyocytes is reduced by between about 4% and 40%, inclusive of each, relative to normal cardiomyocyte physiological function. In some embodiments, the physiological function of a subject's endogenous cardiomyocytes is reduced by between about 5% and 50%, inclusive of each, relative to normal cardiomyocyte physiological function.

In embodiments, the heart disease or condition is pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, other cardiomyopathy, myocarditis, myocardial infarction, myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, or cardiovascular disease. In some embodiments, the heart disease or condition is myocardial infarction (MI).

In some embodiments, the subject is a candidate for a left ventricular assist device (LVAD). In some embodiments, the subject is a candidate for a LVAD at the time of administration of the cardiomyocyte cell therapy, a dose of tacrolimus, and/or a dose of MPA, a pharmaceutical salt thereof (e.g. MS), or a proform thereof (e.g. MMF). In some embodiments, the subject is a candidate for a LVAD at the time of administration of the cardiomyocyte cell therapy. In some embodiments, the subject is a candidate for a LVAD at the time of administration a dose of tacrolimus, and/or a dose of MPA, a pharmaceutical salt thereof (e.g. MS), or a proform thereof (e.g. MMF). In some embodiments, the subject is a candidate for a LVAD at the time of administration a dose of tacrolimus. In some embodiments, the subject is a candidate for a LVAD at the time of administration a dose of MPA, a pharmaceutical salt thereof (e.g. MS), or a proform thereof (e.g. MMF). In some embodiments, the subject is a candidate for a LVAD at the time of administration of the cardiomyocyte cell therapy, a dose of tacrolimus, and a dose of MPA, a pharmaceutical salt thereof (e.g. MS), or a proform thereof (e.g. MMF).

In some embodiments, the subject has a left ventricular assist device (LVAD). In some embodiments, the subject has a LVAD at the time of administration of the cardiomyocyte cell therapy, a dose of tacrolimus, and/or a dose of MPA, a pharmaceutical salt thereof (e.g. MS), or a proform thereof (e.g. MMF). In some embodiments, the subject has a LVAD at the time of administration of the cardiomyocyte cell therapy. In some embodiments, the subject has a LVAD at the time of administration of a dose of tacrolimus, and/or a dose of MPA, a pharmaceutical salt thereof (e.g. MS), or a proform thereof (e.g. MMF). In some embodiments, the subject has a LVAD at the time of administration of a dose of tacrolimus. In some embodiments, the subject has a LVAD at the time of administration of a dose of MPA, a pharmaceutical salt thereof (e.g. MS), or a proform thereof (e.g. MMF). In some embodiments, the subject has a LVAD at the time of administration of the cardiomyocyte cell therapy, a dose of tacrolimus, and a dose of MPA, a pharmaceutical salt thereof (e.g. MS), or a proform thereof (e.g. MMF).

In some embodiments, the subject receives a LVAD at a time subsequent to administration of the cardiomyocyte cell therapy, a dose of tacrolimus, and/or a dose of MPA, a pharmaceutical salt thereof (e.g. MS), or a proform thereof (e.g. MMF). In some embodiments, the subject receives a LVAD at a time subsequent to administration of the cardiomyocyte cell therapy. In some embodiments, the subject receives a LVAD at a time subsequent to administration of a dose of tacrolimus, and/or a dose of MPA, a pharmaceutical salt thereof (e.g. MS), or a proform thereof (e.g. MMF). In some embodiments, the subject receives a LVAD at a time subsequent to administration of a dose of tacrolimus. In some embodiments, the subject receives a LVAD at a time subsequent to administration of a dose of MPA, a pharmaceutical salt thereof (e.g. MS), or a proform thereof (e.g. MMF). In some embodiments, the subject receives a LVAD at a time subsequent to administration of the cardiomyocyte cell therapy, a dose of tacrolimus, and a dose of MPA, a pharmaceutical salt thereof (e.g. MS), or a proform thereof (e.g. MMF).

In some embodiments, the subject is a non-human primate (NHP). In some embodiments, the subject is a human. In some embodiments, the cardiomyocytes are human or are human-derived. Thus, in some embodiments, the provided methods include administration of a cardiomyocyte cell therapy comprising human cardiomyocytes to a human subject.

In some embodiments, the provided methods and uses provide for or achieve improved persistence of administered cardiomyocytes, as compared to certain alternative methods, such as in particular groups of subjects treated, such as in patients with a heart disease or condition, such as myocardial infarction (MI). In some embodiments, the provided methods and uses provide for or achieve fewer side effects, as compared to certain alternative methods, such as in particular groups of subjects treated, such as in patients with a heart disease or condition, such as myocardial infarction (MI). In some embodiments, the methods are advantageous by virtue of administering a cardiomyocyte cell therapy, tacrolimus, and MPA, a pharmaceutical salt thereof (e.g. MS), and/or a proform thereof (e.g. MMF).

In some embodiments, the subject has been previously treated with a therapy or a therapeutic agent for the heart disease or condition, e.g., myocardial infarction (MI), prior to administration of the cardiomyocyte cell therapy.

For the attenuation or prevention of transplant rejection, the appropriate dosage of cardiomyocytes, tacrolimus, and/or MPA, a pharmaceutical salt thereof (e.g. MS), and/or a proform thereof (e.g. MMF), may depend on the type of disease or condition to be treated, the particular cells to be administered, the severity and course of the disease or condition, route of administration, whether the cardiomyocytes, tacrolimus, and/or MPA, a pharmaceutical salt thereof (e.g. MS), and/or a proform thereof (e.g. MMF) are administered for preventive or therapeutic purposes, previous therapy, frequency of administration, the subject's clinical history and response to the cells, and the discretion of the attending physician. The compositions and cells are in some embodiments suitably administered to the subject at one time or over a series of treatments. Exemplary dosage regimens and schedules for the provided methods are described.

In some embodiments, the cardiomyocyte cell therapy, tacrolimus, and MPA, a pharmaceutical salt thereof (e.g. MS), and/or a proform thereof (e.g. MMF) are administered as part of a further combination treatment, which can be administered concurrent with or sequentially to, in any order, another therapeutic intervention. In some contexts, the cardiomyocyte cell therapy is co-administered with another therapy sufficiently close in time such that the composition enhances the effect of one or more additional therapeutic agents, or vice versa. In some embodiments, the cardiomyocyte cell therapy is are administered prior to the one or more additional therapeutic agents. In some embodiments, the cardiomyocyte cell therapy is administered after the one or more additional therapeutic agents. Uses include uses of the cardiomyocyte cell therapy, tacrolimus, and MPA, a pharmaceutical salt thereof (e.g. MS), and/or a proform thereof (e.g. MMF) in such methods and treatments, and uses of such compositions in the preparation of a medicament in order to carry out such methods. In some embodiments, the methods and uses thereby attenuate or prevent rejection of the cardiomyocyte cell therapy administered to the subject.

In some embodiments, the cardiomyocyte cell therapy, tacrolimus, and MPA, a pharmaceutical salt thereof (e.g. MS), and/or a proform thereof (e.g. MMF) are administered without any other treatment.

In some embodiments, prior to, during or following administration of the cardiomyocyte cell therapy, tacrolimus, and MPA, a pharmaceutical salt thereof (e.g. MS), and/or a proform thereof (e.g. MMF), the biological health and/or activity of the cardiomyocyte of the cell therapy is measured, e.g., by any of a number of known methods. Parameters to assess include the viability of the cardiomyocytes, persistence of administered cardiomyocytes, and other measures of cardiomyocyte health and activity, such as measured using any suitable method known in the art. In some embodiments, administration of the cardiomyocyte cell therapy and/or tacrolimus and MPA, a pharmaceutical salt thereof (e.g. MS), and/or a proform thereof (e.g. MMF) and/or any repeated administration of the same, can be determined based on the results of the assays before, during, during the course of or after administration of the cardiomyocyte cell therapy, tacrolimus, and/or MPA, a pharmaceutical salt thereof (e.g. MS), and/or a proform thereof (e.g. MMF).

In some embodiments, tacrolimus and MPA, a pharmaceutical salt thereof (e.g. MS), and/or a proform thereof (e.g. MMF) increase the persistence and/or proliferation of cardiomyocytes administered to the subject. In some embodiments, cardiomyocytes administered to the subject persist for at least one month, at least two months, at least three months, at least four months, at least six months, at least 12 months, at least 18 months, at least 2 years, at least 3 years, at least 4 years, at least 5 years, or more. In some embodiments, the cardiomyocytes administered to the subject persist for at least about six months. In some embodiments, the cardiomyocytes administered to the subject persist for at least about one year. In some embodiments, the cardiomyocytes administered to the subject persist for at least about two years. In some embodiments, the cardiomyocytes administered to the subject persist for at least about three years. In some embodiments, the cardiomyocytes administered to the subject persist for at least about four years. In some embodiments, the cardiomyocytes administered to the subject persist for at least about five years.

A. Administration of an Immunosuppressive Therapy

The provided methods, combinations, kits and uses involve administration of an immunosuppressive therapy of tacrolimus and mycophenolic acid (MPA), a pharmaceutical salt thereof (e.g. MS), and/or a proform thereof (e.g. MMF), each of which can be administered prior to, subsequently to, during, concurrently with, sequentially with and/or intermittently with administration of a cardiomyocyte cell therapy, e.g., a pharmaceutical composition comprising cardiomyocytes (e.g. allogeneic cardiomyocytes) and a pharmaceutically acceptable carrier, and/or the administration of each of which can begin prior to administration of the cardiomyocyte cell therapy and continue until the initiation of administration of the cardiomyocyte cell therapy or after the initiation of administration of the cardiomyocyte cell therapy. In some embodiments, the administration (e.g. daily administration) of tacrolimus and mycophenolic acid (MPA), a pharmaceutical salt thereof (e.g. MS), and/or a proform thereof (e.g. MMF) begins prior to administration of the cardiomyocyte cell therapy (e.g. about 5 days prior) and is continued thereafter, such as continuously throughout the lifespan of the cardiomyocyte cell therapy in the subject. In some embodiments, the immunosuppressive therapy of tacrolimus and MPA, a pharmaceutical salt thereof (e.g. MS), and/or a proform thereof (e.g. MMF) decreases a subject's immune response to administered allogeneic cardiomyocytes.

Tacrolimus, also known as Prograf®, is described in European Patent Application Nos. 0184162 and 0444659 and U.S. Pat. Nos. 6,387,918; 8,486,993; 8,591,946; 8,623,410; 8,664,239; 9,757,362; 9,763,920; 10,166,190; and 10,548,880, which are each incorporated by reference in their entireties. In some embodiments, tacrolimus has the following chemical structure:

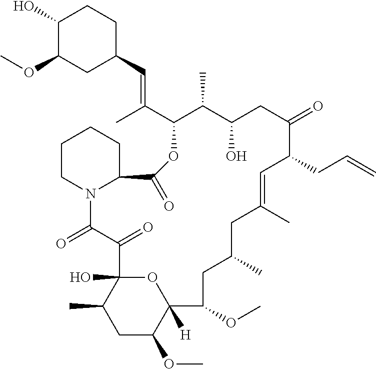 [SMILES: C=CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC]

In some embodiments, mycophenolic acid (MPA) has the following chemical structure:

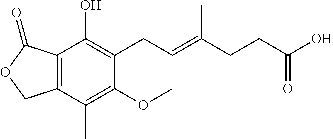 [SMILES: COc1c(C)c2c(c(O)c1C/C=C(\C)CCC(=O)O)C(=O)OC2]

In some embodiments, MPA, a pharmaceutical salt thereof, and/or a proform thereof is the pharmaceutical salt of MPA known as mycophenolate sodium (MS; also known as Myfortic®). MS is described in U.S. Pat. Nos. 6,025,391; 6,172,107; and 6,306,900, which are each incorporated by reference in their entireties. In some embodiments, a pharmaceutical salt of MPA has the following chemical structure:

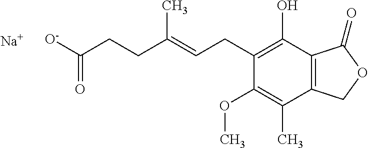 [SMILES: [Na+] ; COc1c(C)c2c(c(O)c1C/C=C(\C)CCC(=O)[O-])C(=O)OC2]

In some embodiments, MPA, a pharmaceutical salt thereof, and/or a proform thereof is the proform of MPA known as mycophenolate mofetil (MMF; also known as CellCept®). MMF is described in U.S. Pat. Nos. 4,753,935; 4,786,637; and 5,543,408, which are each incorporated by reference in their entireties. In some embodiments, a proform of MPA has the following chemical structure:

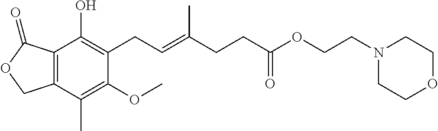 [SMILES: COc1c(C)c2c(c(O)c1C/C=C(\C)CCC(=O)OCCN1CCOCC1)C(=O)OC2]

1. Tacrolimus

a. Compositions and Formulations

In some embodiments of the methods, combinations, kits and uses provided herein, the therapy can be administered in one or more compositions, e.g., a pharmaceutical composition containing tacrolimus.

In some embodiments, the composition, e.g., a pharmaceutical composition containing tacrolimus, can include carriers such as a diluent, adjuvant, excipient, or vehicle with which tacrolimus is administered. Examples of suitable pharmaceutical carriers are described in “Remington's Pharmaceutical Sciences” by E. W. Martin. Such compositions will contain a therapeutically effective amount of tacrolimus, generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Saline solutions and aqueous dextrose and glycerol solutions also can be employed as liquid carriers, particularly for injectable solutions. The pharmaceutical compositions can contain any one or more of a diluents(s), adjuvant(s), antiadherent(s), binder(s), coating(s), filler(s), flavor(s), color(s), lubricant(s), glidant(s), preservative(s), detergent(s), sorbent(s), emulsifying agent(s), pharmaceutical excipient(s), pH buffering agent(s), or sweetener(s) and a combination thereof. In some embodiments, the pharmaceutical composition can be liquid, solid, a lyophilized powder, in gel form, and/or combination thereof. In some aspects, the choice of carrier is determined in part by the particular inhibitor and/or by the method of administration.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), stabilizers and/or preservatives. Compositions containing tacrolimus can also be lyophilized.

In some embodiments, the pharmaceutical composition can be formulated for administration by any route known to those of skill in the art including intramuscular, intravenous, intradermal, intralesional, intraperitoneal injection, subcutaneous, intratumoral, epidural, nasal, oral, vaginal, rectal, topical, local, otic, inhalational, buccal (e.g., sublingual), and transdermal administration or any route. In some embodiments, other modes of administration also are contemplated. In some embodiments, the administration is by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, administration is by parenteral, intrapulmonary, and intranasal, and, if desired for local treatment, intralesional administration. Parenteral infusions include intramuscular, intravenous, intraarterial, intraperitoneal, or subcutaneous administration.

In some embodiments, compositions also can be administered with other biologically active agents, either sequentially, intermittently or in the same composition. In some embodiments, administration also can include controlled release systems including controlled release formulations and device-controlled release, such as by means of a pump. In some embodiments, the administration is oral. In some embodiments, the administration is intravenous.

In some embodiments, tacrolimus is typically formulated and administered in unit dosage forms or multiple dosage forms. Each unit dose contains a predetermined quantity of therapeutically active tacrolimus, sufficient to produce the desired therapeutic effect, in association with the required pharmaceutical carrier, vehicle or diluent. In some embodiments, unit dosage forms, include, but are not limited to, tablets, capsules, pills, powders, granules, sterile parenteral solutions or suspensions, and oral solutions or suspensions, and oil water emulsions containing suitable quantities of tacrolimus. Unit dose forms can be contained ampoules and syringes or individually packaged tablets or capsules. Unit dose forms can be administered in fractions or multiples thereof. In some embodiments, a multiple dose form is a plurality of identical unit dosage forms packaged in a single container to be administered in segregated unit dose form. Examples of multiple dose forms include vials, bottles of tablets or capsules or bottles of pints or gallons.

b. Dosing

In some embodiments, the provided methods involve administering to the subject an immunosuppression therapy of tacrolimus and mycophenolic acid (MPA), a pharmaceutical salt thereof (e.g. MS), and/or a proform thereof (e.g. MMF) and a cardiomyocyte cell therapy.

In some embodiments, the provided methods involve administration of at least one dose of tacrolimus prior to, subsequently to, during, during the course of, concurrent with, sequentially with, and/or intermittently with administration of the cardiomyocyte cell therapy. In some embodiments, the provided methods involve administration of a first dose of tacrolimus prior to, subsequently to, during, during the course of, concurrent with, sequentially with, or intermittently with administration of the cardiomyocyte cell therapy. In some embodiments, “concurrently” indicates that the administration of tacrolimus and that of the cardiomyocyte cell therapy overlap with each other, in that at least one dose of tacrolimus overlaps with administration of the cardiomyocyte cell therapy, and/or that the administration of tacrolimus occurs at the same time (e.g. same day and/or simultaneously) as administration of the cardiomyocyte cell therapy.

In some embodiments, the methods comprise administering at least one dose of tacrolimus prior to, concurrent with, and/or subsequent to administration of the cardiomyocyte cell therapy to the subject.

In some embodiments, administering tacrolimus comprises administering at least one dose of tacrolimus prior to administration of the cardiomyocyte cell therapy to the subject. In some embodiments, at least one dose of tacrolimus is administered about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to administration of the cardiomyocyte cell therapy to the subject.

In some embodiments, at least one dose of tacrolimus is administered about 7 days prior to administration of the cardiomyocyte cell therapy to the subject. In some embodiments, at least one dose of tacrolimus is administered about 6 days prior to administration of the cardiomyocyte cell therapy to the subject. In some embodiments, at least one dose of tacrolimus is administered about 5 days prior to administration of the cardiomyocyte cell therapy to the subject. In some embodiments, at least one dose of tacrolimus is administered about 4 days prior to administration of the cardiomyocyte cell therapy to the subject. In some embodiments, at least one dose of tacrolimus is administered about 3 days prior to administration of the cardiomyocyte cell therapy to the subject. In some embodiments, at least one dose of tacrolimus is administered about 2 days prior to administration of the cardiomyocyte cell therapy to the subject. In some embodiments, at least one dose of tacrolimus is administered about 24 hours prior to administration of the cardiomyocyte cell therapy to the subject. In some embodiments, at least one dose of tacrolimus is administered about 12 hours prior to administration of the cardiomyocyte cell therapy to the subject. In some embodiments, at least one dose of tacrolimus is administered about 10 hours prior to administration of the cardiomyocyte cell therapy to the subject. In some embodiments, at least one dose of tacrolimus is administered about 8 hours prior to administration of the cardiomyocyte cell therapy to the subject. In some embodiments, at least one dose of tacrolimus is administered about 6 hours prior to administration of the cardiomyocyte cell therapy to the subject. In some embodiments, at least one dose of tacrolimus is administered about 4 hours prior to administration of the cardiomyocyte cell therapy to the subject. In some embodiments, at least one dose of tacrolimus is administered about 2 hours prior to administration of the cardiomyocyte cell therapy to the subject. In some embodiments, at least one dose of tacrolimus is administered about 1 hour prior to administration of the cardiomyocyte cell therapy to the subject.

In some embodiments, administering tacrolimus comprises administering at least one dose of tacrolimus concurrent with administration of the cardiomyocyte cell therapy to the subject. In some embodiments, a first dose of tacrolimus is administered to the subject concurrent with administration of the cardiomyocyte cell therapy to the subject.

In some embodiments, administering tacrolimus comprises administering at least one dose of tacrolimus subsequent to administration of the cardiomyocyte cell therapy to the subject. In some embodiments, the at least one dose of tacrolimus is administered to the subject about 1 hour subsequent to, about 2 hours subsequent to, about 4 hours subsequent to, about 6 hours subsequent to, about 8 hours subsequent to, about 10 hours subsequent to, about 12 hours, or about 24 hours subsequent to administration of the cardiomyocyte cell therapy to the subject. In some embodiments, the at least one dose of tacrolimus is administered to the subject about 1 hour subsequent to administration of the cardiomyocyte cell therapy to the subject. In some embodiments, the at least one dose of tacrolimus is administered to the subject about 2 hours subsequent to administration of the cardiomyocyte cell therapy to the subject. In some embodiments, the at least one dose of tacrolimus is administered to the subject about 4 hours subsequent to administration of the cardiomyocyte cell therapy to the subject. In some embodiments, the at least one dose of tacrolimus is administered to the subject about 6 hours subsequent to administration of the cardiomyocyte cell therapy to the subject. In some embodiments, the at least one dose of tacrolimus is administered to the subject about 8 hours subsequent to administration of the cardiomyocyte cell therapy to the subject. In some embodiments, the at least one dose of tacrolimus is administered to the subject about 10 hours subsequent to administration of the cardiomyocyte cell therapy to the subject. In some embodiments, the at least one dose of tacrolimus is administered to the subject about 12 hours subsequent to administration of the cardiomyocyte cell therapy to the subject. In some embodiments, the at least one dose of tacrolimus is administered to the subject about 24 hours subsequent to administration of the cardiomyocyte cell therapy to the subject.

In some embodiments, the methods comprise administering a first dose of tacrolimus prior to, concurrent with, and/or subsequent to administration of the cardiomyocyte cell therapy to the subject.

In some embodiments administering tacrolimus comprises administering a first dose of tacrolimus prior to administration of the cardiomyocyte cell therapy to the subject. In some embodiments, a first dose of tacrolimus is administered about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to administration of the cardiomyocyte cell therapy to the subject.

In some embodiments, a first dose of tacrolimus is administered to the subject about 7 days prior to administration of the cardiomyocyte cell therapy to the subject. In some embodiments, a first dose of tacrolimus is administered to the subject about 6 days prior to administration of the cardiomyocyte cell therapy to the subject. In some embodiments, a first dose of tacrolimus is administered to the subject about 5 days prior to administration of the cardiomyocyte cell therapy to the subject. In some embodiments, a first dose of tacrolimus is administered to the subject about 4 days prior to administration of the cardiomyocyte cell therapy to the subject. In some embodiments, a first dose of tacrolimus is administered to the subject about 3 days prior to administration of the cardiomyocyte cell therapy to the subject. In some embodiments, a first dose of tacrolimus is administered to the subject about 2 days prior to administration of the cardiomyocyte cell therapy to the subject. In some embodiments, a first dose of tacrolimus is administered to the subject about 24 hours prior to administration of the cardiomyocyte cell therapy to the subject. In some embodiments, a first dose of tacrolimus is administered to the subject about 12 hours prior to administration of the cardiomyocyte cell therapy to the subject. In some embodiments, a first dose of tacrolimus is administered to the subject about 10 hours prior to administration of the cardiomyocyte cell therapy to the subject. In some embodiments, a first dose of tacrolimus is administered to the subject about 8 hours prior to administration of the cardiomyocyte cell therapy to the subject. In some embodiments, a first dose of tacrolimus is administered to the subject about 6 hours prior to administration of the cardiomyocyte cell therapy to the subject. In some embodiments, a first dose of tacrolimus is administered to the subject about 4 hours prior to administration of the cardiomyocyte cell therapy to the subject. In some embodiments, a first dose of tacrolimus is administered to the subject about 2 hours prior to administration of the cardiomyocyte cell therapy to the subject. In some embodiments, a first dose of tacrolimus is administered to the subject about 1 hour prior to administration of the cardiomyocyte cell therapy to the subject.

In some embodiments, a first dose of tacrolimus is administered to the subject subsequent to administration of the cardiomyocyte cell therapy to the subject. In some embodiments, a first dose of tacrolimus is administered to the subject about 1 hour subsequent to, about 2 hours subsequent to, about 4 hours subsequent to, about 6 hours subsequent to, about 8 hours subsequent to, about 10 hours subsequent to, about 12 hours, or about 24 hours subsequent to administration of the cardiomyocyte cell therapy to the subject. In some embodiments, a first dose of tacrolimus is administered to the subject about 1 hour subsequent to administration of the cardiomyocyte cell therapy to the subject. In some embodiments, a first dose of tacrolimus is administered to the subject about 2 hours subsequent to administration of the cardiomyocyte cell therapy to the subject. In some embodiments, a first dose of tacrolimus is administered to the subject about 4 hours subsequent to administration of the cardiomyocyte cell therapy to the subject. In some embodiments, a first dose of tacrolimus is administered to the subject about 6 hours subsequent to administration of the cardiomyocyte cell therapy to the subject. In some embodiments, a first dose of tacrolimus is administered to the subject about 8 hours subsequent to administration of the cardiomyocyte cell therapy to the subject. In some embodiments, a first dose of tacrolimus is administered to the subject about 10 hours subsequent to administration of the cardiomyocyte cell therapy to the subject. In some embodiments, a first dose of tacrolimus is administered to the subject about 12 hours subsequent to administration of the cardiomyocyte cell therapy to the subject. In some embodiments, a first dose of tacrolimus is administered to the subject about 24 hours subsequent to administration of the cardiomyocyte cell therapy to the subject. In some embodiments, a first dose of tacrolimus is administered to the subject no sooner than about 2 hours subsequent to administration of the cardiomyocyte cell therapy to the subject.

In some embodiments, the total daily dosage of tacrolimus administered to the subject yields a blood trough level of between about 1 ng/mL and about 30 ng/mL, between about 2 ng/mL and about 25 ng/mL, between about 5 ng/mL and about 20 ng/mL, or between about 10 ng/mL and about 15 ng/mL, inclusive of each. In some embodiments, the total daily dosage of tacrolimus administered to the subject yields a blood trough level of between about 1 ng/mL and about 30 ng/mL, inclusive of each. In some embodiments, the total daily dosage of tacrolimus administered to the subject yields a blood trough level of between about 2 ng/mL and about 25 ng/mL, inclusive of each. In some embodiments, the total daily dosage of tacrolimus administered to the subject yields a blood trough level of between about 5 ng/mL and about 20 ng/mL, inclusive of each. In some embodiments, the total daily dosage of tacrolimus administered to the subject yields a blood trough level of between about 10 ng/mL and about 15 ng/mL, inclusive of each. In some embodiments, the total daily dosage of tacrolimus administered to the subject yields a blood trough level of between about 5 ng/mL and about 10 ng/mL, inclusive of each. In some embodiments, the total daily dosage of tacrolimus administered to the subject yields a blood trough level of about 5 ng/mL. In some embodiments, the total daily dosage of tacrolimus administered to the subject yields a blood trough level of about 10 ng/mL. In some embodiments, the total daily dosage of tacrolimus administered to the subject yields a blood trough level of about 15 ng/mL.

In some embodiments, the total daily dosage of tacrolimus administered to the subject is between about 0.05 kg/mg and about 0.3 mg/kg, between about 0.075 mg/kg and about 0.25 mg/kg, between about 0.1 mg/kg and about 0.225 mg/kg, or between about 0.15 mg/kg and about 0.20 mg/kg, inclusive of each. In some embodiments, total daily dosage of tacrolimus administered to the subject is between about 0.05 kg/mg and about 0.3 mg/kg, inclusive of each. In some embodiments, total daily dosage of tacrolimus administered to the subject is between about 0.075 mg/kg and about 0.25 mg/kg, inclusive of each. In some embodiments, total daily dosage of tacrolimus administered to the subject is between about 0.1 mg/kg and about 0.225 mg/kg, inclusive of each. In some embodiments, the total daily dosage of tacrolimus administered to the subject is between about 0.15 mg/kg and about 0.20 mg/kg, inclusive of each. In some embodiments, the total daily dosage of tacrolimus administered to the subject is about 0.075 mg/kg. In some embodiments, the total daily dosage of tacrolimus administered to the subject is about 0.1 mg/kg. In some embodiments, the total daily dosage of tacrolimus administered to the subject is about 0.15 mg/kg. In some embodiments, the total daily dosage of tacrolimus administered to the subject is about 0.2 mg/kg.

In some embodiments, tacrolimus is administered daily. In some embodiments, tacrolimus is administered at least once daily. In some embodiments, the total daily dosage of tacrolimus is provided as a single dose per day. In some embodiments, the total daily dosage of tacrolimus is divided between 2 doses, 3 doses, or 4 doses per day. In some embodiments, the total daily dosage of tacrolimus is divided between 2 doses per day. In some embodiments, a dose of tacrolimus is administered about every 12 hours. In some embodiments, the total daily dosage of tacrolimus is divided between 3 doses per day. In some embodiments, the total daily dosage of tacrolimus is divided between 4 doses per day.

In some embodiments, tacrolimus is administered daily for about 3 months, about 6 months, about 12 months, about 24 months, about 36 months, about 48 months, about 60 months, or more subsequent to administration of the cardiomyocyte cell therapy to the subject. In some embodiments, tacrolimus is administered daily for about 3 months. In some embodiments, tacrolimus is administered daily for about 6 months. In some embodiments, tacrolimus is administered daily for about 9 months. In some embodiments, tacrolimus is administered daily for about 12 months. In some embodiments, tacrolimus is administered daily for about 24 months. In some embodiments, tacrolimus is administered daily for about 48 months. In some embodiments, tacrolimus is administered daily for about 60 months.

In some embodiments, tacrolimus is administered daily for the lifetime of the cardiomyocyte cell therapy in the subject.

In some embodiments, tacrolimus is administered prior to and subsequent to administration of the cardiomyocyte cell therapy to the subject.

In some embodiments, tacrolimus is administered prior to, concurrent with, and subsequent to administration of the cardiomyocyte cell therapy to the subject.

In some embodiments, a first dose of tacrolimus is administered to the subject about 5 days prior to administration of the cardiomyocyte cell therapy to the subject; and two doses of tacrolimus are administered daily to the subject (bid), beginning at the first dose of tacrolimus and continuously thereafter for the lifetime of the administered cardiomyocyte cell therapy. In some embodiments, a dose of tacrolimus is administered to the subject about every 12 hours.

In some embodiments, administration of tacrolimus comprises oral, subcutaneous, and/or intravenous administration. In some embodiments administration of tacrolimus comprises subcutaneous administration. In some embodiments administration of tacrolimus comprises intravenous administration. In some embodiments administration of tacrolimus comprises oral administration. In some embodiments administration of tacrolimus comprises intravenous administration and oral administration.

2. Mycophenolic Acid and Pharmaceutical Salts and Proforms Thereof

The provided methods, combinations, kits and uses involve administration of an immunosuppressive therapy of tacrolimus and mycophenolic acid (MPA), a pharmaceutical salt thereof (e.g. MS), and/or a proform thereof (e.g. MMF). In some embodiments, a pharmaceutical salt of MPA is mycophenolate sodium (MS). In some embodiments, a proform of MPA is mycophenolate mofetil (MMF).

a. Compositions and Formulations

In some embodiments of the methods, combinations, kits and uses provided herein, the therapy can be administered in one or more compositions, e.g., a pharmaceutical composition containing MPA, a proform thereof (e.g. mycophenolate mofetil; MMF), and/or a pharmaceutical salt thereof (e.g. mycophenolate sodium; MS).

In some embodiments, the composition, e.g., a pharmaceutical composition containing MPA, a pharmaceutical salt thereof (e.g. MS), and/or a proform thereof (e.g. MMF), can include carriers such as a diluent, adjuvant, excipient, or vehicle with which MPA, a pharmaceutical salt thereof (e.g. MS), and/or a proform thereof (e.g. MMF) is administered. Examples of suitable pharmaceutical carriers are described in “Remington's Pharmaceutical Sciences” by E. W. Martin. Such compositions will contain a therapeutically effective amount of MPA, a pharmaceutical salt thereof (e.g. MS), and/or a proform thereof (e.g. MMF), generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Saline solutions and aqueous dextrose and glycerol solutions also can be employed as liquid carriers, particularly for injectable solutions. The pharmaceutical compositions can contain any one or more of a diluents(s), adjuvant(s), antiadherent(s), binder(s), coating(s), filler(s), flavor(s), color(s), lubricant(s), glidant(s), preservative(s), detergent(s), sorbent(s), emulsifying agent(s), pharmaceutical excipient(s), pH buffering agent(s), or sweetener(s) and a combination thereof. In some embodiments, the pharmaceutical composition can be liquid, solid, a lyophilized powder, in gel form, and/or combination thereof. In some aspects, the choice of carrier is determined in part by the particular compound and/or by the method of administration.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), stabilizers and/or preservatives. Compositions containing MPA, a pharmaceutical salt thereof (e.g. MS), and/or a proform thereof (e.g. MMF) can also be lyophilized.

In some embodiments, the pharmaceutical composition can be formulated for administration by any route known to those of skill in the art including intramuscular, intravenous, intradermal, intralesional, intraperitoneal injection, subcutaneous, intratumoral, epidural, nasal, oral, vaginal, rectal, topical, local, otic, inhalational, buccal (e.g., sublingual), and transdermal administration or any route. In some embodiments, other modes of administration also are contemplated. In some embodiments, the administration is by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, administration is by parenteral, intrapulmonary, and intranasal, and, if desired for local treatment, intralesional administration. Parenteral infusions include intramuscular, intravenous, intraarterial, intraperitoneal, or subcutaneous administration.

In some embodiments, compositions also can be administered with other biologically active agents, either sequentially, intermittently or in the same composition. In some embodiments, administration also can include controlled release systems including controlled release formulations and device controlled release, such as by means of a pump. In some embodiments, the administration is oral. In some embodiments, the administration is intravenous.

In some embodiments, MPA, a pharmaceutical salt thereof (e.g. MS), and/or a proform thereof (e.g. MMF) is typically formulated and administered in unit dosage forms or multiple dosage forms. Each unit dose contains a predetermined quantity of therapeutically active MPA, a pharmaceutical salt thereof (e.g. MS), and/or a proform thereof (e.g. MMF) sufficient to produce the desired therapeutic effect, in association with the required pharmaceutical carrier, vehicle or diluent. In some embodiments, unit dosage forms, include, but are not limited to, tablets, capsules, pills, powders, granules, sterile parenteral solutions or suspensions, and oral solutions or suspensions, and oil water emulsions containing suitable quantities of MPA, a pharmaceutical salt thereof (e.g. MS), and/or a proform thereof (e.g. MMF). Unit dose forms can be contained ampoules and syringes or individually packaged tablets or capsules. Unit dose forms can be administered in fractions or multiples thereof. In some embodiments, a multiple dose form is a plurality of identical unit dosage forms packaged in a single container to be administered in segregated unit dose form. Examples of multiple dose forms include vials, bottles of tablets or capsules or bottles of pints or gallons.

a. Dosing

In some embodiments, the provided methods involve administering to the subject an immunosuppression therapy of tacrolimus and mycophenolic acid (MPA), a pharmaceutical salt thereof (e.g. MS), and/or a proform thereof (e.g. MMF) and a cardiomyocyte cell therapy.

In some embodiments, the provided methods involve administration of at least one dose of MPA, a pharmaceutical salt thereof (e.g. MS), and/or a proform thereof (e.g. MMF) prior to, subsequently to, during, during the course of, concurrent with, sequentially with, and/or intermittently with administration of the cardiomyocyte cell therapy. In some embodiments, the provided methods involve administration of a first dose of MPA, a pharmaceutical salt thereof (e.g. MS), and/or a proform thereof (e.g. MMF) prior to, subsequently to, during, during the course of, concurrent with, sequentially with, or intermittently with administration of the cardiomyocyte cell therapy. In some embodiments, “concurrently” indicates that the administration of MPA, a pharmaceutical salt thereof (e.g. MS), and/or a proform thereof (e.g. MMF) and that of the cardiomyocyte cell therapy overlap with each other, in that at least one dose of MPA, a pharmaceutical salt thereof (e.g. MS), and/or a proform thereof (e.g. MMF) overlaps with administration of the cardiomyocyte cell therapy, and/or that the administration of MPA, a pharmaceutical salt thereof (e.g. MS), and/or a proform thereof (e.g. MMF) occurs at the same time (e.g. same day and/or simultaneously) as administration of the cardiomyocyte cell therapy.

In some embodiments, the methods comprise administering at least one dose of MPA, a pharmaceutical salt thereof (e.g. MS), and/or a proform thereof (e.g. MMF) prior to, concurrent with, and/or subsequent to administration of the cardiomyocyte cell therapy to the subject.

In some embodiments, the at least one dose of MPA, a pharmaceutical salt there of (e.g. MS), and/or a proform thereof (e.g. MMF) is administered about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to administration of the cardiomyocyte cell therapy to the subject. In some embodiments, the at least one dose of MPA, a pharmaceutical salt thereof (e.g. MS), and/or a proform thereof (e.g. MMF) is administered about 7 days prior to administration of the cardiomyocyte cell therapy to the subject. In some embodiments, the at least one dose of MPA, a pharmaceutical salt thereof (e.g. MS), and/or a proform thereof (e.g. MMF) is administered about 6 days prior to administration of the cardiomyocyte cell therapy to the subject. In some embodiments, the at least one dose of MPA, a pharmaceutical salt thereof (e.g. MS), and/or a proform thereof (e.g. MMF) is administered about 5 days prior to administration of the cardiomyocyte cell therapy to the subject. In some embodiments, the at least one dose of MPA, a pharmaceutical salt thereof (e.g. MS), and/or a proform thereof (e.g. MMF) is administered about 4 days prior to administration of the cardiomyocyte cell therapy to the subject. In some embodiments, the at least one dose of MPA, a pharmaceutical salt thereof (e.g. MS), and/or a proform thereof (e.g. MMF) is administered about 3 days prior to administration of the cardiomyocyte cell therapy to the subject. In some embodiments, the at least one dose of MPA, a pharmaceutical salt thereof (e.g. MS), and/or a proform thereof (e.g. MMF) is administered about 2 days prior to administration of the cardiomyocyte cell therapy to the subject. In some embodiments, the at least one dose of MPA, a pharmaceutical salt thereof (e.g. MS), and/or a proform thereof (e.g. MMF) is administered about 24 hours prior to administration of the cardiomyocyte cell therapy to the subject. In some embodiments, the at least one dose of MPA, a pharmaceutical salt thereof (e.g. MS), and/or a proform thereof (e.g. MMF) is administered about 12 hours prior to administration of the cardiomyocyte cell therapy to the subject. In some embodiments, the at least one dose of MPA, a pharmaceutical salt thereof (e.g. MS), and/or a proform thereof (e.g. MMF) is administered about 10 hours prior to administration of the cardiomyocyte cell therapy to the subject. In some embodiments, the at least one dose of MPA, a pharmaceutical salt thereof (e.g. MS), and/or a proform thereof (e.g. MMF) is administered about 8 hours prior to administration of the cardiomyocyte cell therapy to the subject. In some embodiments, the at least one dose of MPA, a pharmaceutical salt thereof (e.g. MS), and/or a proform thereof (e.g. MMF) is administered about 6 hours prior to administration of the cardiomyocyte cell therapy to the subject. In some embodiments, the at least one dose of MPA, a pharmaceutical salt thereof (e.g. MS), and/or a proform thereof (e.g. MMF) is administered about 4 hours prior to administration of the cardiomyocyte cell therapy to the subject. In some embodiments, the at least one dose of MPA, a pharmaceutical salt thereof (e.g. MS), and/or a proform thereof (e.g. MMF) is administered about 2 hours prior to administration of the cardiomyocyte cell therapy to the subject. In some embodiments, the at least one dose of MPA, a pharmaceutical salt thereof (e.g. MS), and/or a proform thereof (e.g. MMF) is administered about 1 hour prior to administration of the cardiomyocyte cell therapy to the subject.

In some embodiments, administering MPA, a pharmaceutical salt thereof (e.g. MS), and/or a proform thereof (e.g. MMF) comprises administering at least one dose of MPA, a pharmaceutical salt thereof (e.g. MS), and/or a proform thereof (e.g. MMF) concurrent with administration of the cardiomyocyte cell therapy to the subject.

In some embodiments, administering MPA, a pharmaceutical salt thereof (e.g. MS), and/or a proform thereof (e.g. MMF) comprises administering at least one dose of MPA, a pharmaceutical salt thereof (e.g. MS), and/or a proform thereof (e.g. MMF) subsequent to administration of the cardiomyocyte cell therapy to the subject. In some embodiments, at least one dose of MPA, a pharmaceutical salt thereof (e.g. MS), and/or a proform thereof (e.g. MMF) is administered to the subject about 1 hour subsequent to, about 2 hours subsequent to, about 4 hours subsequent to, about 6 hours subsequent to, about 8 hours subsequent to, about 10 hours subsequent to, about 12 hours, or about 24 hours subsequent to administration of the cardiomyocyte cell therapy to the subject. In some embodiments, at least one dose of MPA, a pharmaceutical salt thereof (e.g. MS), and/or a proform thereof (e.g. MMF) is administered to the subject about 1 hour subsequent to administration of the cardiomyocyte cell therapy to the subject. In some embodiments, at least one dose of MPA, a pharmaceutical salt thereof (e.g. MS), and/or a proform thereof (e.g. MMF) is administered to the subject about 2 hours subsequent to administration of the cardiomyocyte cell therapy to the subject. In some embodiments, at least one dose of MPA, a pharmaceutical salt thereof (e.g. MS), and/or a proform thereof (e.g. MMF) is administered to the subject about 4 hours subsequent to administration of the cardiomyocyte cell therapy to the subject. In some embodiments, at least one dose of MPA, a pharmaceutical salt thereof (e.g. MS), and/or a proform thereof (e.g. MMF) is administered to the subject about 6 hours subsequent to administration of the cardiomyocyte cell therapy to the subject. In some embodiments, at least one dose of MPA, a pharmaceutical salt thereof (e.g. MS), and/or a proform thereof (e.g. MMF) is administered to the subject about 8 hours subsequent to administration of the cardiomyocyte cell therapy to the subject. In some embodiments, at least one dose of MPA, a pharmaceutical salt thereof (e.g. MS), and/or a proform thereof (e.g. MMF) is administered to the subject about 10 hours subsequent to administration of the cardiomyocyte cell therapy to the subject. In some embodiments, at least one dose of MPA, a pharmaceutical salt thereof (e.g. MS), and/or a proform thereof (e.g. MMF) is administered to the subject about 12 hours subsequent to administration of the cardiomyocyte cell therapy to the subject. In some embodiments, at least one dose of MPA, a pharmaceutical salt thereof (e.g. MS), and/or a proform thereof (e.g. MMF) is administered to the subject about 24 hours subsequent to administration of the cardiomyocyte cell therapy to the subject.

In some embodiments, the methods comprise administering a first dose of MPA, a pharmaceutical salt thereof (e.g. MS), and/or a proform thereof (e.g. MMF) prior to, concurrent with, and/or subsequent to administration of the cardiomyocyte cell therapy to the subject.

In some embodiments, a first dose of MPA, a pharmaceutical salt thereof (e.g. MS), and/or a proform thereof (e.g. MMF) is administered about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to administration of the cardiomyocyte cell therapy to the subject. In some embodiments, a first dose of MPA, a pharmaceutical salt thereof (e.g. MS), and/or a proform thereof (e.g. MMF) is administered about 7 days prior to administration of the cardiomyocyte cell therapy to the subject. In some embodiments, a first dose of MPA, a pharmaceutical salt thereof (e.g. MS), and/or a proform thereof (e.g. MMF) is administered about 6 days prior to administration of the cardiomyocyte cell therapy to the subject. In some embodiments, a first dose of MPA, a pharmaceutical salt thereof (e.g. MS), and/or a proform thereof (e.g. MMF) is administered about 5 days prior to administration of the cardiomyocyte cell therapy to the subject. In some embodiments, a first dose of MPA, a pharmaceutical salt thereof (e.g. MS), and/or a proform thereof (e.g. MMF) is administered about 4 days prior to administration of the cardiomyocyte cell therapy to the subject. In some embodiments, a first dose of MPA, a pharmaceutical salt thereof (e.g. MS), and/or a proform thereof (e.g. MMF) is administered about 3 days prior to administration of the cardiomyocyte cell therapy to the subject. In some embodiments, a first dose of MPA, a pharmaceutical salt thereof (e.g. MS), and/or a proform thereof (e.g. MMF) is administered about 2 days prior to administration of the cardiomyocyte cell therapy to the subject. In some embodiments, a first dose of MPA, a pharmaceutical salt thereof (e.g. MS), and/or a proform thereof (e.g. MMF) is administered about 24 hours prior to administration of the cardiomyocyte cell therapy to the subject. In some embodiments, a first dose of MPA, a pharmaceutical salt thereof (e.g. MS), and/or a proform thereof (e.g. MMF) is administered about 12 hours prior to administration of the cardiomyocyte cell therapy to the subject. In some embodiments, a first dose of MPA, a pharmaceutical salt thereof (e.g. MS), and/or a proform thereof (e.g. MMF) is administered about 10 hours prior to administration of the cardiomyocyte cell therapy to the subject. In some embodiments, a first dose of MPA, a pharmaceutical salt thereof (e.g. MS), and/or a proform thereof (e.g. MMF) is administered about 8 hours prior to administration of the cardiomyocyte cell therapy to the subject. In some embodiments, a first dose of MPA, a pharmaceutical salt thereof (e.g. MS), and/or a proform thereof (e.g. MMF) is administered about 6 hours prior to administration of the cardiomyocyte cell therapy to the subject. In some embodiments, a first dose of MPA, a pharmaceutical salt thereof (e.g. MS), and/or a proform thereof (e.g. MMF) is administered about 4 hours prior to administration of the cardiomyocyte cell therapy to the subject. In some embodiments, a first dose of MPA, a pharmaceutical salt thereof (e.g. MS), and/or a proform thereof (e.g. MMF) is administered about 2 hours prior to administration of the cardiomyocyte cell therapy to the subject. In some embodiments, a first dose of MPA, a pharmaceutical salt thereof (e.g. MS), and/or a proform thereof (e.g. MMF) is administered about 1 hour prior to administration of the cardiomyocyte cell therapy to the subject.

In some embodiments, administering MPA, a pharmaceutical salt thereof (e.g. MS), and/or a proform thereof (e.g. MMF) comprises administering a first dose of MPA, a pharmaceutical salt thereof (e.g. MS), and/or a proform thereof (e.g. MMF) concurrent with administration of the cardiomyocyte cell therapy to the subject.

In some embodiments, administering MPA, a pharmaceutical salt thereof (e.g. MS), and/or a proform thereof (e.g. MMF) comprises administering a first dose of MPA, a pharmaceutical salt thereof (e.g. MS), and/or a proform thereof (e.g. MMF) subsequent to administration of the cardiomyocyte cell therapy to the subject. In some embodiments, a first dose of MPA, a pharmaceutical salt thereof (e.g. MS), and/or a proform thereof (e.g. MMF) is administered to the subject about 1 hour subsequent to, about 2 hours subsequent to, about 4 hours subsequent to, about 6 hours subsequent to, about 8 hours subsequent to, about 10 hours subsequent to, about 12 hours, or about 24 hours subsequent to administration of the cardiomyocyte cell therapy to the subject. In some embodiments, a first dose of MPA, a pharmaceutical salt thereof (e.g. MS), and/or a proform thereof (e.g. MMF) is administered to the subject about 1 hour subsequent to administration of the cardiomyocyte cell therapy to the subject. In some embodiments, a first dose of MPA, a pharmaceutical salt thereof (e.g. MS), and/or a proform thereof (e.g. MMF) is administered to the subject about 2 hours subsequent to administration of the cardiomyocyte cell therapy to the subject. In some embodiments, a first dose of MPA, a pharmaceutical salt thereof (e.g. MS), and/or a proform thereof (e.g. MMF) is administered to the subject about 4 hours subsequent to administration of the cardiomyocyte cell therapy to the subject. In some embodiments, a first dose of MPA, a pharmaceutical salt thereof (e.g. MS), and/or a proform thereof (e.g. MMF) is administered to the subject about 6 hours subsequent to administration of the cardiomyocyte cell therapy to the subject. In some embodiments, a first dose of MPA, a pharmaceutical salt thereof (e.g. MS), and/or a proform thereof (e.g. MMF) is administered to the subject about 8 hours subsequent to administration of the cardiomyocyte cell therapy to the subject. In some embodiments, a first dose of MPA, a pharmaceutical salt thereof (e.g. MS), and/or a proform thereof (e.g. MMF) is administered to the subject about 10 hours subsequent to administration of the cardiomyocyte cell therapy to the subject. In some embodiments, a first dose of MPA, a pharmaceutical salt thereof (e.g. MS), and/or a proform thereof (e.g. MMF) is administered to the subject about 12 hours subsequent to administration of the cardiomyocyte cell therapy to the subject. In some embodiments, a first dose of MPA, a pharmaceutical salt thereof (e.g. MS), and/or a proform thereof (e.g. MMF) is administered to the subject about 24 hours subsequent to administration of the cardiomyocyte cell therapy to the subject.

In some embodiments, MPA, a pharmaceutical salt thereof (e.g. MS), and/or a proform thereof (e.g. MMF) is administered daily. In some embodiments, MPA, a pharmaceutical salt thereof (e.g. MS), and/or a proform thereof (e.g. MMF) is administered at least once daily. In some embodiments, the total daily dosage of MPA, a pharmaceutical salt thereof (e.g. MS), and/or a proform thereof (e.g. MMF) is provided as a single dose per day. In some embodiments, the total daily dosage of MPA, a pharmaceutical salt thereof (e.g. MS), and/or a proform thereof (e.g. MMF) is divided between two doses, three doses, or four doses per day.

In some embodiments, the total daily dosage of MPA, a pharmaceutical salt thereof (e.g. MS), and/or a proform thereof (e.g. MMF) is divided between two doses per day (bid). In some embodiments, a dose of MPA, a pharmaceutical salt thereof (e.g. MS), and/or a proform thereof (e.g. MMF) is administered about every 12 hours. In some embodiments, the total daily dosage of MPA, a pharmaceutical salt thereof (e.g. MS), and/or a proform thereof (e.g. MMF) is divided between three doses per day. In some embodiments, the total daily dosage of MPA, a pharmaceutical salt thereof (e.g. MS), and/or a proform thereof (e.g. MMF) is divided between four doses per day.

In some embodiments, MPA, a pharmaceutical salt thereof (e.g. MS), and/or a proform thereof (e.g. MMF) is administered daily for about 3 months, about 6 months, about 12 months, about 24 months, about 36 months, about 48 months, about 60 months, or more subsequent to administration of the cardiomyocyte cell therapy to the subject. In some embodiments, MPA, a pharmaceutical salt thereof (e.g. MS), and/or a proform thereof (e.g. MMF) is administered daily for about 3 months subsequent to administration of the cardiomyocyte cell therapy to the subject. In some embodiments, MPA, a pharmaceutical salt thereof (e.g. MS), and/or a proform thereof (e.g. MMF) is administered daily for about 6 months subsequent to administration of the cardiomyocyte cell therapy to the subject. In some embodiments, MPA, a pharmaceutical salt thereof (e.g. MS), and/or a proform thereof (e.g. MMF) is administered daily for about 12 months subsequent to administration of the cardiomyocyte cell therapy to the subject. In some embodiments, MPA, a pharmaceutical salt thereof (e.g. MS), and/or a proform thereof (e.g. MMF) is administered daily for about 24 months subsequent to administration of the cardiomyocyte cell therapy to the subject. In some embodiments, MPA, a pharmaceutical salt thereof (e.g. MS), and/or a proform thereof (e.g. MMF) is administered daily for about 36 months subsequent to administration of the cardiomyocyte cell therapy to the subject. In some embodiments, MPA, a pharmaceutical salt thereof (e.g. MS), and/or a proform thereof (e.g. MMF) is administered daily for about 48 months subsequent to administration of the cardiomyocyte cell therapy to the subject. In some embodiments, MPA, a pharmaceutical salt thereof (e.g. MS), and/or a proform thereof (e.g. MMF) is administered daily for about 60 months subsequent to administration of the cardiomyocyte cell therapy to the subject.

In some embodiments, MPA, a pharmaceutical salt thereof (e.g. MS), and/or a proform thereof (e.g. MMF) is administered daily for the lifetime of the cardiomyocyte cell therapy in the subject.

In some embodiments, MPA, a pharmaceutical salt thereof (e.g. MS), and/or a proform thereof (e.g. MMF) is administered prior to and subsequent to administration of the cardiomyocyte cell therapy to the subject.

In some embodiments, MPA, a pharmaceutical salt thereof (e.g. MS), and/or a proform thereof (e.g. MMF) is administered prior to, concurrent with, and subsequent to administration of the cardiomyocyte cell therapy to the subject.

In some embodiments, a first dose of MPA or a pharmaceutical salt or a proform thereof (e.g. MMF or MS) is administered to the subject about 5 days prior to administration of the cardiomyocyte cell therapy to the subject; and two doses of MPA or a pharmaceutical salt or a proform thereof (e.g. MMF or MS) are administered daily to the subject (bid), beginning at the first dose of MPA, a pharmaceutical salt thereof (e.g. MS), and/or a proform thereof (e.g. MMF) and continuously thereafter for the lifetime of the administered cardiomyocyte cell therapy. In some embodiments, a dose of MPA, a pharmaceutical salt thereof (e.g. MS), and/or a proform thereof (e.g. MMF) is administered to the subject about every 12 hours.

In some embodiments, a first dose of tacrolimus and a first dose of MPA, a pharmaceutical salt thereof (e.g. MS), and/or a proform thereof (e.g. MMF) are each administered to the subject on the same day. In some embodiments, tacrolimus and MPA, a pharmaceutical salt thereof (e.g. MS), and/or a proform thereof (e.g. MMF) are each administered to the subject daily.

In some embodiments, the total daily dosage of tacrolimus is divided between two doses per day and the total daily dosage of MPA, a pharmaceutical salt thereof (e.g. MS), and/or a proform thereof (e.g. MMF) is divided between two doses per day.

In some embodiments, a first dose of tacrolimus and a first dose of MPA, a pharmaceutical salt thereof (e.g. MS), and/or a proform thereof (e.g. MMF) are each administered to the subject about 5 days prior to administration of the cardiomyocyte cell therapy to the subject; and two doses of tacrolimus and two doses of MPA, a pharmaceutical salt thereof (e.g. MS), and/or a proform thereof (e.g. MMF) are administered daily to the subject (bid) beginning at the first dose of tacrolimus and MPA, a pharmaceutical salt thereof (e.g. MS), and/or a proform thereof (e.g. MMF) and continuously thereafter for the lifetime of the administered cardiomyocyte cell therapy. In some embodiments, two doses of tacrolimus are administered about every 12 hours. In some embodiments, two doses of MPA, a pharmaceutical salt thereof (e.g. MS), and/or a proform thereof (e.g. MMF) are administered about every 12 hours. In some embodiments, two doses of tacrolimus and two doses of MPA, a pharmaceutical salt thereof (e.g. MS), and/or a proform thereof (e.g. MMF) are administered about every 12 hours.

In some embodiments, MPA, a pharmaceutical salt thereof, or a proform thereof is mycophenolate mofetil (MMF). In some embodiments, the total daily dosage of MMF is between about 500 mg and about 3000 mg, between about 1000 mg and about 2500 mg, or between about 1500 mg and about 2000 mg, inclusive of each. In some embodiments, the total daily dosage of MMF is between about 500 mg and about 3000 mg, inclusive of each. In some embodiments, the total daily dosage of MMF is between about 1000 mg and about 2500 mg, inclusive of each. In some embodiments, the total daily dosage of MMF is between about 1500 mg and about 2000 mg, inclusive of each.

In some embodiments, the total daily dosage of MMF is about 1000 mg, about 1500 mg, about 2000 mg, or about 2500 mg. In some embodiments, the total daily dosage of MMF is about 1000 mg. In some embodiments, the total daily dosage of MMF is about 1500 mg. In some embodiments, the total daily dosage of MMF is about 2000 mg. In some embodiments, the total daily dosage of MMF is about 2500 mg.

In some embodiments, MMF is administered orally. In some embodiments, (i) administration of tacrolimus includes subcutaneous administration and (ii) administration of MMF includes oral administration.

If a subject cannot tolerate oral administration of MMF, MMF can be alternatively provided by intravenous administration. Thus, in some embodiments, MMF is administered intravenously. In some embodiments, (i) administration of tacrolimus includes subcutaneous administration and (ii) administration of MMF includes intravenous administration.

Methods for preparing and administering MMF (CellCept®) orally and intravenously are known in the art. For example, intravenous MMF (CellCept®) is recommended for subjects unable to tolerate oral MMF (CellCept®) and can be administered at the same dose as oral MMF (CellCept®). See Pescovitz et al., Clin. Transplantation (2000) 14:179-88. In some cases, a subject that cannot tolerate oral MMF (CellCept®) is administered intravenous MMF (CellCept®) until the subject can tolerate oral MMF (CellCept®), at which point the subject is switched from intravenous to oral MMF (CellCept®). Thus, in some cases, administration of MMF comprises intravenous administration followed by oral administration.

In some embodiments, MPA, a pharmaceutical salt thereof, or a proform thereof is mycophenolate sodium (MS). In some embodiments, the total daily dosage of MS is between about 360 mg and about 2700 mg, between about 720 mg and about 2160 mg, or between about 720 mg and about 1620 mg, inclusive of each. In some embodiments, the total daily dosage of MS is between about 360 mg and about 2700 mg, inclusive of each. In some embodiments, the total daily dosage of MS is between about 720 mg and about 2160 mg, inclusive of each. In some embodiments, the total daily dosage of MS is between about 720 mg and about 1620 mg, inclusive of each. In some embodiments, the total daily dosage of MS is about 360 mg, about 720 mg, about 1080 mg, or about 1440 mg. In some embodiments, the total daily dosage of MS is about 360 mg. In some embodiments, the total daily dosage of MS is about 720 mg. In some embodiments, the total daily dosage of MS is about 1080 mg. In some embodiments, the total daily dosage of MS is about 1440 mg.

In some embodiments, MPA, a pharmaceutical salt thereof, or a proform thereof is mycophenolate mofetil (MMF) and mycophenolate sodium (MS).

B. Administration of a Cardiomyocyte Cell Therapy

Provided herein are methods of administering and uses of an immunosuppressive therapy of tacrolimus and mycophenolic acid (MPA), a pharmaceutical salt thereof (e.g. mycophenolate sodium; MS), and/or a proform thereof (e.g. mycophenolate mofetil; MMF), in combination with administration of a cardiomyocyte cell therapy to a subject in need thereof, wherein the immunosuppressive therapy attenuates or prevents rejection of the cardiomyocyte cell therapy administered to the subject.

Methods for administration of cardiomyocyte compositions are known and may be used in connection with the provided methods and compositions.

In some embodiments, administration of the cardiomyocyte cell therapy comprises delivery into a subject's heart tissue. In some embodiments, delivery into a subject's heart tissue comprises intravenous injection, intraarterial injection, intracoronary injection, intramuscular injection, intraperitoneal injection, intramyocardial injection, trans-endocardial injection, trans-epicardial injection, and/or infusion. In some embodiments, delivery into a subject's heart tissue comprises intramyocardial injection. In some embodiments, delivery into a subject's heart tissue comprises trans-epicardial injection. In some embodiments, delivery into a subject's heart tissue comprises trans-endocardial injection. In some embodiments, delivery into a subject's heart tissue comprises delivery at the site of a myocardial infarct (MI). In some embodiments, delivery into a subject's heart tissue comprises delivery near the site of a myocardial infarct (MI). In some embodiments, delivery into a subject's heart tissue comprises delivery within about 1 mm, 5 mm, 10 mm, 20 mm, 30 mm, 40 mm, 50 mm, 60 mm, 70 mm, 80 mm, or 90 mm of the site of an injury, such as a MI. In some embodiments, delivery into a subject's heart tissue comprises delivery within about 0.1 cm, 0.2 cm, 0.3 cm, 0.4 cm, 0.5 cm, 0.6 cm, 0.7 cm, 0.8 cm, 0.9 cm, 1.0 cm, 1.2 cm, 1.4 cm, 1.6 cm, 1.8 cm, 2.0 cm, 2.5 cm, 3.0 cm, 3.5 cm, 4.0 cm, 4.5 cm, or 5.0 of the site of an injury such as a MI.

In some embodiments, the cardiomyocyte cell therapy is a pharmaceutical composition comprising cardiomyocytes and a pharmaceutically acceptable carrier. In some embodiments the pharmaceutically acceptable carrier comprises cell culture medium. In some embodiments, the pharmaceutical acceptable carrier is cell culture medium. In some embodiments, the pharmaceutically acceptable carrier comprises between about 1% and 20% of the total volume of the cardiomyocyte cell therapy composition. In some embodiments, the pharmaceutically acceptable carrier comprises between about 5% and 10% of the total volume of the cardiomyocyte cell therapy composition. In some embodiments, the pharmaceutically acceptable carrier comprises about 5% of the total volume of the cardiomyocyte cell therapy composition. In some embodiments, the pharmaceutically acceptable carrier comprises about 10% of the total volume of the cardiomyocyte cell therapy composition. In some embodiments, the pharmaceutically acceptable carrier comprises about 15% of the total volume of the cardiomyocyte cell therapy composition.

In some embodiments, the cardiomyocyte cell therapy is a suspension of cardiomyocytes. In some embodiments, the cardiomyocyte cell therapy is an engineered tissue graft comprising cardiomyocytes.

In any of the provided embodiments, the subject administered the cardiomyocyte cell therapy has a condition or disease, such as a heart condition or disease. In some embodiments, the heart condition or disease is selected from the group consisting of pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, other cardiomyopathy, myocarditis, myocardial infarction (MI), myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, or cardiovascular disease. In some embodiments, the heart condition or disease is myocardial infarction (MI). Thus, in some embodiments, the cardiomyocyte cell therapy is administered to a subject to treat a MI (e.g. as a composition comprising cardiomyocytes).

In some embodiments, the subject is a candidate for a left ventricular assist device (LVAD). In some embodiments, the is a candidate for a LVAD at the time of administration of the cardiomyocyte cell therapy, a dose of tacrolimus, and/or a dose of MPA, a pharmaceutical salt thereof (e.g. MS), and/or a proform thereof (e.g. MMF). In some embodiments, the subject is a candidate for a LVAD at the time of administration of the cardiomyocyte cell therapy. In some embodiments, the subject has a left ventricular assist device (LVAD). In some embodiments, the subject has a left ventricular assist device (LVAD) at the time of administration of the cardiomyocyte cell therapy, a dose of tacrolimus, and/or a dose of MPA, a pharmaceutical salt thereof (e.g. MS), and/or a proform thereof (e.g. MMF). In some embodiments, the subject has a LVAD at the time of administration of the cardiomyocyte cell therapy. In some embodiments, the subject receives a LVAD at a time subsequent to administration of the cardiomyocyte cell therapy, a dose of tacrolimus, and/or a dose of MPA, a pharmaceutical salt thereof (e.g. MS), and/or a proform thereof (e.g. MMF). In some embodiments, the subject receives a LVAD at a time subsequent to administration of the cardiomyocyte cell therapy.

In some embodiments, the subject is a human. In some embodiments, the subject is a non-human primate (NHP).

### II. CARDIOMYOCYTE COMPOSITIONS AND GENERATION THEREOF

Provided herein are cardiomyocyte cell therapy compositions for use with an immunosuppressive therapy of tacrolimus and mycophenolic acid (MPA), a pharmaceutical salt thereof (e.g. mycophenolate sodium; MS), and/or a proform thereof (e.g. mycophenolate mofetil; MMF), to attenuate or prevent rejection of the cardiomyocyte cell therapy administered to a subject in need thereof.

In some embodiments, the cardiomyocyte cell therapy is a pharmaceutical composition comprising cardiomyocytes and a pharmaceutically acceptable carrier. In some embodiments, the cardiomyocyte cell therapy is a suspension of cardiomyocytes. In some embodiments, the cardiomyocyte cell therapy is an engineered tissue graft comprising cardiomyocytes.

In some embodiments, the cardiomyocytes of the cardiomyocyte cell therapy are primary cardiomyocytes derived from a donor, such as a human donor. In some embodiments, the primary cardiomyocytes are allogeneic to the recipient.

In some embodiments, the cardiomyocytes of the cardiomyocyte cell therapy are derived from pluripotent stem cells (PSCs). In some embodiments, the cardiomyocytes are differentiated from PSCs, such as induced PSCs (iPSCs). In some cases, the cardiomyocytes are differentiated from iPSCs derived from a donor, such as a human donor.

Soluble factors important for embryonic cardiac development include Activin A, BMP4, nodal, Wnt agonists and antagonists, bFGF and other molecules (Conlon et al, Development 120(7): 1919 (1994); Lough et al, Dev. Biol. (1996) 178(1): 198; Mima et al, PNAS (1995) 92(2):467; Zaffran and Frasch, Circ. Res. (2002) 91 (6), 457). Any suitable method of inducing cardiomyocyte differentiation may be used, for example, any of those described in Fujiwara et al., PLoS One. (2001) 6(2):e16734; Dambrot et al., Biochem J. (2011) 434(1):25-35; Foldes et al., J Mol Cell Cardiol. (2011) 50(2):367-76; Wang et al., Sci China Life Sci. (2010) 53(5):581-9; Chen et al., J Cell Biochem. (2010) (1):29-39; Yang et al., Nature (2008) 453:524-28; Kattman et al., Cell Stem Cell (2011) 8:228-40; Laflamme et al., Nat. Biotechnol. (2007) 25: 1015-24; Paige et al., PLoS One (2010) 5(6): e11134; Xu et al., Regen Med (2011) 6(1):53-66; Mignone et al., Circ J (2010) 74(12):2517-26; and Takei et al., Am J Physiol Heart Circ Physiol. (2009) 296(6):H1793-803, each herein incorporated by reference in its entirety. PSCs (e.g., iPSCs or ESCs) can also be differentiated into cardiomyocytes by any of the methods described in WO2013013206 and WO2013056072, each incorporated by reference in its entirety.

A variety of different methods of generating pluripotent stem cells (generally referred to as iPSCs; miPSCs for murine cells or hiPSCs for human cells) are known. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al, World J. Stem Cells 7(1): 116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more “reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogenous genes. This loss of the episomal vector(s) results in cells that are called “zero footprint” cells. This is desirable as the fewer genetic modifications (particularly in the genome of the host cell), the better. Thus, it is preferred that the resulting hiPSCs have no permanent genetic modifications.

The number of reprogramming factors that can be used or are used can vary. Commonly, when fewer reprogramming factors are used, the efficiency of the transformation of the cells to a pluripotent state goes down, as well as the “pluripotency”, e.g. fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT; SOX2, OCT4, (POU5F1), KLF4, MYC, NANOG, LIN28, and SV40L T antigen.

In general, these reprogramming factor genes are provided on episomal vectors such as are known in the art and commercially available. For example, ThermoFisher/Invitrogen sell a sendai virus reprogramming kit for zero footprint generation of hiPSCs, see catalog number A34546. ThermoFisher also sells EBNA-based systems as well, see catalog number A14703.

In addition, there are a number of commercially available hiPSC lines available; see, e.g., the Gibco® Episomal hiPSC line, K18945, which is a zero footprint, viral-integration-free human iPSC cell line (see also Burridge et al, 2011, supra).

In general, iPSCs are made from non-pluripotent cells such as CD34+ cord blood cells, fibroblasts, etc., by transiently expressing the reprogramming factors as described herein. For example, successful iPSCs were also generated using only Oct3/4, Sox2 and Klf4, while omitting the C-Myc, although with reduced reprogramming efficiency.

In general, iPSCs are characterized by the expression of certain factors that include KLF4, Nanog, OCT4, SOX2, ESRRB, TBX3, c-Myc and TCL1. New or increased expression of these factors for purposes of the invention may be via induction or modulation of an endogenous locus or from expression from a transgene.

For example, murine iPSCs can be generated using the methods of Diecke et al, Sci Rep. 2015, Jan. 28; 5:8081 (doi: 10.1038/srep08081), hereby incorporated by reference in its entirety and specifically for the methods and reagents for the generation of the miPSCs. See also, e.g., Burridge et al., PLoS One, 2011 6(4): 18293, hereby incorporated by reference in its entirety and specifically for the methods outlined therein.

In some embodiments, PSCs (e.g. iPSCs) generated by any of the methods described herein and/or known in the art are differentiated into cardiomyocytes, such as to produce a composition highly enriched in cardiomyocytes.

The PSCs (e.g. iPSCs) can be differentiated into cardiomyocytes by any known methods, including but not limited to those described in Murry and Keller, Cell (2008) 132(4):661-80; Burridge et al., Cell Stem Cell (2012) 10:16-28; Lian et al., Nature Protocols (2013) 8:162-65; Batalov and Feiberg, Biomark. Insight (2015) 10(Suppl. 1):71-6; Denning et al., Biochim. Biophys. Acta Mol. Cell Res. (2016) 1863:1728-48; Breckwoldt et al., Nature Protocols (2017) 12:1177-97; Guo et al., Stem Cell Res. And Ther. (2018) 9:44; and Leitolis et al., Front. Cell Dev. Biol. (2019) 8:164.

In some embodiments, the cardiomyocytes are allogeneic to a subject receiving a transplant of the cardiomyocytes. Thus, in some embodiments, the PSCs (e.g. iPSCs) from which cardiomyocytes are derived are engineered to be hypoimmunogenic by any known methods.

For example, nucleic acid sequences may be modified within PSCs (e.g. iPSCs) to generate hypoimmunogenic PSCs. Technologies to modify nucleic acid sequences within cells include homologous recombination, knock-in, knock-out, ZFNs (zinc finger nucleases), TALENs (transcription activator-like effector nucleases), CRISPR (clustered regularly interspaced short palindromic repeats)/Cas9, and other site-specific nuclease technologies. These techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

A number of different techniques can be used to engineer the PSCs (e.g. iPSCs) to be hypoimmunogenic, including those described in WO 2020/018615, incorporated herein by reference in its entirety. In some embodiments, engineering of the PSCs (iPSCs) to be hypoimmunogenic reduces an immune response of the recipient to the cells, including cardiomyocytes differentiated from the hypoimmunogenic PSCs (e.g. iPSCs).

### III. ARTICLES OF MANUFACTURE AND KITS

Also provided are articles of manufacture containing tacrolimus, mycophenolic acid (MPA), a pharmaceutical salt thereof (e.g. MS), and/or a proform thereof (e.g. MMF), and/or a cardiomyocyte cell therapy, and/or compositions thereof. The articles of manufacture may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing a disease or condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition.

The article of manufacture may include (a) a first container with a composition contained therein, wherein the composition is a cardiomyocyte cell therapy (e.g. a pharmaceutical composition comprising cardiomyocytes and a pharmaceutically acceptable carrier); (b) a second container with a first composition and a second composition contained therein, wherein the first composition includes tacrolimus and the second composition includes MPA, a pharmaceutical salt thereof (e.g. MS), and/or a proform thereof (e.g. MMF).

The article of manufacture may include (a) a first container with a composition contained therein, wherein the composition is a cardiomyocyte cell therapy, (e.g. a pharmaceutical composition comprising cardiomyocytes and a pharmaceutically acceptable carrier); (b) a second container with a composition contained therein, wherein the composition includes tacrolimus; and (c) a third container with a composition contained therein, wherein the composition includes MPA, a pharmaceutical salt thereof (e.g. MS), and/or a proform thereof (e.g. MMF).

The article of manufacture may further include a package insert indicating that the compositions can be used to treat or prevent a particular condition (e.g. rejection of a cardiomyocyte cell therapy). Alternatively, or additionally, the article of manufacture may further include another or the same container comprising a pharmaceutically-acceptable buffer or excipient. It may further include other materials such as other buffers, diluents, filters, needles, and/or syringes.

### IV. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, “treatment” (and grammatical variations thereof such as “treat” or “treating”) refers to complete or partial amelioration or reduction of rejection of a cardiomyocyte cell therapy or a symptom, adverse effect or outcome, or phenotype associated therewith. Desirable effects of treatment include, but are not limited to, preventing occurrence or recurrence of rejection of a cardiomyocyte cell therapy, alleviation of symptoms, diminishment of any direct or indirect pathological consequences of the rejection, preventing rejection of a cardiomyocyte cell therapy, decreasing the rate of such rejection, amelioration or palliation of the rejection, and cessation of rejection or improved prognosis. The terms do not imply complete prevention or cure of rejection or complete elimination of any symptom or effect(s) on all symptoms or outcomes.

“Preventing,” as used herein, includes providing prophylaxis with respect to the occurrence or recurrence of rejection of a cardiomyocyte cell therapy in a subject that may be predisposed to the rejection but has not yet been diagnosed with the rejection. In some embodiments, the methods described herein prevent the onset of rejection of a cardiomyocyte cell therapy.

“Attenuating,” as used herein, includes reducing the severity of, reducing, and/or delaying the onset of rejection of a cardiomyocyte cell therapy. In some embodiments, the methods described herein attenuate rejection of a cardiomyocyte cell therapy.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation, cells, or composition refers to an amount effective, at dosages and for periods of time necessary, to achieve a desired therapeutic result, such as for treatment of a disease, condition, or disorder (e.g. rejection of a cardiomyocyte cell therapy), and/or pharmacokinetic or pharmacodynamics effect of the treatment. The therapeutically effective amount may vary according to factors such as the disease or condition state, age, sex, and weight of the subject, and the populations of cells administered. In some embodiments, the provided methods involve administering the molecules, cells, and/or compositions at effective amounts, e.g., therapeutically effective amounts.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease or condition (e.g. rejection of a cardiomyocyte cell therapy), the prophylactically effective amount will be less than the therapeutically effective amount.

As used herein, a “subject” or an “individual” is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject is human.

The term “pluripotent cells” refers to cells that can self-renew and proliferate while remaining in an undifferentiated state and that can, under the proper conditions, be induced to differentiate into specialized cell types. The term “pluripotent cells,” as used herein, encompasses embryonic stem cells and other types of stem cells, including fetal, amnionic, or somatic stem cells. Exemplary human stem cell lines include the H9 human embryonic stem cell line. Additional exemplary stem cell lines include those made available through the National Institutes of Health Human Embryonic Stem Cell Registry and the Howard Hughes Medical Institute HUES collection (as described in Cowan, C. A. et. al, New England J. Med. 350: 13. (2004), incorporated by reference herein in its entirety.)

“Pluripotent stem cells” as used herein have the potential to differentiate into any of the three germ layers: endoderm, mesoderm, or ectoderm. The term “pluripotent stem cells,” as used herein, also encompasses “induced pluripotent stem cells”, or “iPSCs”, a type of pluripotent stem cell derived from a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art and are further described below. (See, e.g., Zhou et al, Stem Cells 27 (11): 2667-74 (2009); Huangfu et al., Nature Biotechnol. 26 (7): 795 (2008); Woltjen et al, Nature 458 (7239): 766-770 (2009); and Zhou et al, Cell Stem Cell 8:381-384 (2009); each of which is incorporated by reference herein in their entirety.) As used herein, “hiPSCs” are human induced pluripotent stem cells, and “riPSCs” are rhesus induced pluripotent stem cells.

“Pluripotent stem cell characteristics” refer to characteristics of a cell that distinguish pluripotent stem cells from other cells. The ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic. Expression or non-expression of certain combinations of molecular markers are also pluripotent stem cell characteristics. For example, human pluripotent stem cells express at least several, and in some embodiments, all of the markers from the following non-limiting list: S SEA-3, S SEA-4, TRA-1-60, TRA-1-81, TRA-2-49/6E, ALP, Sox2, E-cadherin, UTF-1, Oct4, Rexl, and Nanog. Cell morphologies associated with pluripotent stem cells are also pluripotent stem cell characteristics.

As used herein, “multipotent” or “multipotent cell” refers to a cell type that can give rise to a limited number of other particular cell types. For example, induced multipotent cells are capable of forming endodermal cells. Additionally, multipotent blood stem cells can differentiate itself into several types of blood cells, including lymphocytes, monocytes, neutrophils, etc.

As used herein, the term “oligopotent” refers to the ability of an adult stem cell to differentiate into only a few different cell types. For example, lymphoid or myeloid stem cells are capable of forming cells of either the lymphoid or myeloid lineages, respectively.

As used herein, the term “unipotent” means the ability of a cell to form a single cell type. For example, spermatogonial stem cells are only capable of forming sperm cells.

As used herein, the term “totipotent” means the ability of a cell to form an entire organism. For example, in mammals, only the zygote and the first cleavage stage blastomeres are totipotent.

As used herein, “non-pluripotent cells” refer to mammalian cells that are not pluripotent cells. Examples of such cells include differentiated cells as well as progenitor cells. Examples of differentiated cells include, but are not limited to, cells from a tissue selected from bone marrow, skin, skeletal muscle, fat tissue and peripheral blood. Exemplary cell types include, but are not limited to, fibroblasts, hepatocytes, myoblasts, neurons, osteoblasts, osteoclasts, and T-cells. The starting cells employed for generating the induced multipotent cells can be non-pluripotent cells.

Differentiated cells include, but are not limited to, multipotent cells, oligopotent cells, unipotent cells, progenitor cells, and terminally differentiated cells. In particular embodiments, a less potent cell is considered “differentiated” in reference to a more potent cell.

A “somatic cell” is a cell forming the body of an organism. Somatic cells include cells making up organs, skin, blood, bones and connective tissue in an organism, but not germ cells.

Cells can be from, for example, human or non-human mammals Exemplary non-human mammals include, but are not limited to, mice, rats, cats, dogs, rabbits, guinea pigs, hamsters, sheep, pigs, horses, bovines, and non-human primates. In some embodiments, a cell is from an adult human or non-human mammal. In some embodiments, a cell is from a neonatal human, an adult human, or non-human mammal.

The “HLA” or “human leukocyte antigen” complex is a gene complex encoding the major histocompatibility complex (MHC) proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A, HLA-B and HLA-C, which present peptides from the inside of the cell, and antigens presented by the HLA-I complex attract killer T-cells (also known as CD8+ T-cells or cytotoxic T cells). The HLA-I proteins are associated with (3-2 microglobulin (β2M). HLA-II includes five proteins, HLA-DP, HLA-DM, HLA-DOB, HLA-DQ and HLA-DR, which present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

By “hypoimmunogenic cell,” herein is meant a cell that gives rise to a reduced immunological rejection response when transferred into an allogeneic host. In preferred embodiments, hypoimmunogenic cells do not give rise to an immune response. Thus, “hypo-immunogenic” or “hypoimmune” refers to a significantly reduced or eliminated immune response when compared to the immune response of a parental (i.e., wild-type” or “wt”) cell prior to immunoengineering as outlined herein.

By “knock out” in the context of a gene means that the host cell harboring the knock out does not produce a functional protein product of the gene. As outlined herein, a knock out can result in a variety of ways, from removing all or part of the coding sequence, introducing frameshift mutations such that a functional protein is not produced (either truncated or nonsense sequence), removing or altering a regulatory component (e.g. a promoter) such that the gene is not transcribed, preventing translation through binding to mRNA, etc. Generally, the knock out is effected at the genomic DNA level, such that the cells' offspring also carry the knock out permanently.

By “knock in” in the context of a gene means that the host cell harboring the knock in has more functional protein active in the cell. As outlined herein, a knock in can be done in a variety of ways, usually by the introduction of at least one copy of a transgene (tg) encoding the protein into the cell, although this can also be done by replacing regulatory components as well, for example by adding a constitutive promoter to the endogenous gene. In general, knock in technologies result in the integration of the extra copy of the transgene into the host cell.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The terms “polypeptide” and “protein” are used interchangeably to refer to a polymer of amino acid residues, and are not limited to a minimum length. Polypeptides, including the antibodies and antibody chains and other peptides may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “package insert” is used to refer to instructions customarily included in commercial packages of therapeutic products, that contain information about the indications, usage, dosage, administration, combination therapy, contraindications and/or warnings concerning the use of such therapeutic products.

A “pharmaceutically acceptable carrier” refers to an ingredient in a pharmaceutical formulation, other than an active ingredient, which is nontoxic to a subject. A pharmaceutically acceptable carrier includes, but is not limited to, a cell culture medium, a buffer, excipient, stabilizer, or preservative.

As used herein, the singular forms “a,” “an,” and “the” include plural referents unless the context clearly dictates otherwise. For example, “a” or “an” means “at least one” or “one or more.” It is understood that aspects, embodiments, and variations described herein include “comprising,” “consisting,” and/or “consisting essentially of” aspects, embodiments and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “composition” refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### V. EXEMPLARY EMBODIMENTS

Among the provided embodiments are:

1. A method of attenuating or preventing rejection of a cardiomyocyte cell therapy in a subject, the method comprising administering to a subject (i) tacrolimus and (ii) mycophenolic acid (MPA), a pharmaceutical salt thereof, and/or a proform thereof, each prior to, concurrent with, and/or subsequent to administration of a cardiomyocyte cell therapy to the subject.
2. A method of attenuating or preventing rejection of a cardiomyocyte cell therapy in a subject, the method comprising administering to a subject a cardiomyocyte cell therapy prior to, concurrent with, and/or subsequent to administration of (i) tacrolimus and (ii) mycophenolic acid (MPA), a pharmaceutical salt thereof, and/or a proform thereof to the subject.
3. A method of attenuating or preventing rejection of a cardiomyocyte cell therapy in a subject, the method comprising:

(1) administering a cardiomyocyte cell therapy to a subject; and

(2) administering (i) tacrolimus and (ii) mycophenolic acid (MPA), a pharmaceutical salt thereof, and/or a proform thereof to the subject, wherein administration of (i) tacrolimus and (ii) MPA, a pharmaceutical salt thereof, and/or a proform thereof is prior to, concurrent with, and/or subsequent to administration of the cardiomyocyte cell therapy to the subject.

4. The method of any of embodiments 1-3, wherein the cardiomyocyte cell therapy is a pharmaceutical composition comprising cardiomyocytes and a pharmaceutically acceptable carrier.
5. The method of any of embodiments 1-4, wherein the cardiomyocyte cell therapy is administered as a suspension of cardiomyocytes or as an engineered tissue graft comprising cardiomyocytes and a matrix.
6. The method of any of embodiments 1-5, wherein the cardiomyocyte cell therapy is administered as a suspension of cardiomyocytes.
7. The method of any of embodiments 1-6, wherein administration of tacrolimus comprises administration of at least one dose of tacrolimus prior to, concurrent with, and/or subsequent to administration of the cardiomyocyte cell therapy to the subject.
8. The method of any of embodiments 1-7, wherein administration of tacrolimus comprises administration of at least one dose of tacrolimus prior to administration of the cardiomyocyte cell therapy to the subject.
9. The method of embodiment 8, wherein the at least one dose of tacrolimus is administered about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to administration of the cardiomyocyte cell therapy to the subject.
10. The method of any of embodiments 1-9, wherein a first dose of tacrolimus is administered to the subject about 5 days prior to administration of the cardiomyocyte cell therapy to the subject.
11. The method of any of embodiments 1-10, wherein administration of tacrolimus comprises administration of at least one dose of tacrolimus concurrent with administration of the cardiomyocyte cell therapy to the subject.
12. The method of any of embodiments 1-7, wherein a first dose of tacrolimus is administered to the subject concurrent with administration of the cardiomyocyte cell therapy to the subject.
13. The method of any of embodiments 1-12, wherein administration of tacrolimus comprises administration of at least one dose of tacrolimus subsequent to administration of the cardiomyocyte cell therapy to the subject.
14. The method of embodiment 13, wherein the at least one dose of tacrolimus is administered to the subject about 1 hour subsequent to, about 2 hours subsequent to, about 4 hours subsequent to, about 6 hours subsequent to, about 8 hours subsequent to, about 10 hours subsequent to, about 12 hours, or about 24 hours subsequent to administration of the cardiomyocyte cell therapy to the subject.
15. The method of any of embodiments 1-7, 13, and 14, wherein a first dose of tacrolimus is administered to the subject subsequent to administration of the cardiomyocyte cell therapy to the subject, optionally about 1 hour subsequent to, about 2 hours subsequent to, about 4 hours subsequent to, about 6 hours subsequent to, about 8 hours subsequent to, about 10 hours subsequent to, about 12 hours, or about 24 hours subsequent to administration of the cardiomyocyte cell therapy to the subject.
16. The method of any of embodiments 1-15, wherein the total daily dosage of tacrolimus administered to the subject yields a blood trough level of between about 1 ng/mL and about 30 ng/mL, between about 2 ng/mL and about 25 ng/mL, between about 5 ng/mL and about 20 ng/mL, or between about 10 ng/mL and about 15 ng/mL, inclusive of each.
17. The method of any of embodiments 1-16, wherein the total daily dosage of tacrolimus administered to the subject yields a blood trough level of between about 5 ng/mL and about 10 ng/mL, inclusive of each.
18. The method of any of embodiments 1-16, wherein the total daily dosage of tacrolimus administered to the subject yields a blood trough level of between about 10 ng/mL and about 15 ng/mL, inclusive of each.
19. The method of any of embodiments 1-18, wherein tacrolimus is administered at least once daily.
20. The method of any of embodiments 1-19, wherein the total daily dosage of tacrolimus is provided as a single dose per day.
21. The method of any of embodiments 1-19, wherein the total daily dosage of tacrolimus is divided between 2 doses, 3 doses, or 4 doses per day.
22. The method of any of embodiments 1-19 and 21, wherein the total daily dosage of tacrolimus is divided between two doses per day (bid), optionally wherein a dose of tacrolimus is administered about every 12 hours.
23. The method of any of embodiments 1-22, wherein tacrolimus is administered daily for about 3 months, about 6 months, about 12 months, about 24 months, about 36 months, about 48 months, about 60 months, or more subsequent to administration of the cardiomyocyte cell therapy to the subject.
24. The method of any of embodiments 1-23, wherein tacrolimus is administered daily for the lifetime of the cardiomyocyte cell therapy in the subject.
25. The method of any of embodiments 1-11, 13-14, 16-19, and 21-24, wherein:

(a) a first dose of tacrolimus is administered to the subject about 5 days prior to administration of the cardiomyocyte cell therapy to the subject; and

(b) two doses of tacrolimus are administered daily to the subject (bid), optionally about every 12 hours, beginning at the first dose of tacrolimus and continuously thereafter for the lifetime of the administered cardiomyocyte cell therapy.

26. The method of any of embodiments 1-25, wherein administration of tacrolimus comprises oral, subcutaneous, and/or intravenous administration.
27. The method of any of embodiments 1-26, wherein administration of tacrolimus comprises oral administration.
28. The method of any of embodiments 1-26, wherein administration of tacrolimus comprises subcutaneous administration.
29. The method of any of embodiments 1-26, wherein administration of tacrolimus comprises intravenous administration.
30. The method of any of embodiments 1-29, wherein administration of MPA, a pharmaceutical salt thereof, and/or a proform thereof comprises administration of at least one dose of MPA, a pharmaceutical salt thereof, and/or a proform thereof prior to, concurrent with, and/or subsequent to administration of the cardiomyocyte cell therapy to the subject.
31. The method of any of embodiments 1-30, wherein administration of MPA, a pharmaceutical salt thereof, and/or a proform thereof comprises administration of at least one dose of MPA, a pharmaceutical salt thereof, and/or a proform thereof prior to administration of the cardiomyocyte cell therapy to the subject.
32. The method of embodiment 31, wherein the at least one dose of MPA, a pharmaceutical salt thereof, and/or a proform thereof is administered about 7 days prior to, about 6 days prior to, about 5 days prior to, about 4 days prior to, about 3 days prior to, about 2 days prior to, about 1 day prior to, about 12 hours prior to, about 10 hours prior to, about 8 hours prior to, about 6 hours prior to, about 4 hours prior to, about 2 hours prior to, or about 1 hour prior to administration of the cardiomyocyte cell therapy to the subject.
33. The method of any of embodiments 1-32, wherein a first dose of MPA, a pharmaceutical salt thereof, and/or a proform thereof is administered to the subject about 5 days prior to administration of the cardiomyocyte cell therapy to the subject.
34. The method of any of embodiments 1-33, wherein administration of MPA, a pharmaceutical salt thereof, and/or a proform thereof comprises administration of at least one dose of MPA, a pharmaceutical salt thereof, and/or a proform thereof concurrent with administration of the cardiomyocyte cell therapy to the subject.
35. The method of any of embodiments 1-32 and 34, wherein a first dose of MPA, a pharmaceutical salt thereof, and/or a proform thereof is administered to the subject concurrent with administration of the cardiomyocyte cell therapy to the subject.
36. The method of any of embodiments 1-35, wherein administration of MPA, a pharmaceutical salt thereof, and/or a proform thereof comprises administration of at least one dose of MPA, a pharmaceutical salt thereof, and/or a proform thereof subsequent to administration of the cardiomyocyte cell therapy to the subject.
37. The method of embodiment 36, wherein the at least one dose of MPA, a pharmaceutical salt thereof, and/or a proform thereof is administered to the subject about 1 hour subsequent to, about 2 hours subsequent to, about 4 hours subsequent to, about 6 hours subsequent to, about 8 hours subsequent to, about 10 hours subsequent to, about 12 hours, or about 24 hours subsequent to administration of the cardiomyocyte cell therapy to the subject.
38. The method of any of embodiments 1-32 and 36-37, wherein a first dose of MPA, a pharmaceutical salt thereof, and/or a proform thereof is administered to the subject subsequent to administration of the cardiomyocyte cell therapy to the subject, optionally about 1 hour subsequent to, about 2 hours subsequent to, about 4 hours subsequent to, about 6 hours subsequent to, about 8 hours subsequent to, about 10 hours subsequent to, about 12 hours, or about 24 hours subsequent to administration of the cardiomyocyte cell therapy to the subject.
39. The method of any of embodiments 1-38, wherein MPA, a pharmaceutical salt thereof, and/or a proform thereof is administered at least once daily.
40. The method of any of embodiments 1-39, wherein the total daily dosage of MPA, a pharmaceutical salt thereof, and/or a proform thereof is provided as a single dose per day.
41. The method of any of embodiments 1-39, wherein the total daily dosage of MPA, a pharmaceutical salt thereof, and/or a proform thereof is divided between two doses, three doses, or four doses per day.
42. The method of any of embodiments 1-39 and 41, wherein the total daily dosage of MPA, a pharmaceutical salt thereof, and/or a proform thereof is divided between two doses per day (bid), optionally wherein a dose of MPA, a pharmaceutical salt thereof, and/or a proform thereof is administered about every 12 hours.
43. The method of any of embodiments 1-42, wherein MPA, a pharmaceutical salt thereof, and/or a proform thereof is administered daily for about 3 months, about 6 months, about 12 months, about 24 months, about 36 months, about 48 months, about 60 months, or more subsequent to administration of the cardiomyocyte cell therapy to the subject.
44. The method of any of embodiments 1-43, wherein MPA, a pharmaceutical salt thereof, and/or a proform thereof is administered daily for the lifetime of the administered cardiomyocyte cell therapy.
45. The method of any of embodiments 1-34, 36-37, 39 and 41-44, wherein:

(a) a first dose of MPA, a pharmaceutical salt thereof, and/or a proform thereof is administered to the subject about 5 days prior to administration of the cardiomyocyte cell therapy to the subject; and

(b) two doses of MPA, a pharmaceutical salt thereof, and/or a proform thereof are administered daily to the subject (bid), optionally about every 12 hours, beginning at the first dose and continuously thereafter for the lifetime of the administered cardiomyocyte cell therapy.

46. The method of any of embodiments 1-45, wherein administration of MPA, a pharmaceutical salt thereof, and/or a proform thereof comprises oral, subcutaneous, and/or intravenous administration.
47. The method of any of embodiments 1-46, wherein administration of MPA, a pharmaceutical salt thereof, and/or a proform thereof comprises oral administration.
48. The method of any of embodiments 1-46, wherein administration of MPA, a pharmaceutical salt thereof, and/or a proform thereof comprises intravenous administration.
49. The method of any of embodiments 1-28, 30-46, and 48, wherein (i) administration of MPA, a pharmaceutical salt thereof, and/or a proform thereof comprises intravenous administration; and (ii) administration of tacrolimus comprises subcutaneous administration.
50. The method of any of embodiments 1-49, wherein a first dose of tacrolimus and a first dose of MPA, a pharmaceutical salt thereof, and/or a proform thereof are each administered to the subject on the same day.
51. The method of any of embodiments 1-50, wherein tacrolimus and MPA, a pharmaceutical salt thereof, and/or a proform thereof are each administered to the subject daily.
52. The method of any of embodiments 1-19, 21-39, and 41-51, wherein the total daily dosage of tacrolimus is divided between two doses per day and the total daily dosage of MPA, a pharmaceutical salt thereof, and/or a proform thereof is divided between two doses per day.
53. The method of any of embodiments 1-11, 13-14, 16-19, 21-34, 36-37, 39, and 41-52, wherein:

(a) a first dose of tacrolimus and a first dose of MPA, a pharmaceutical salt thereof, and/or a proform thereof are each administered to the subject about 5 days prior to administration of the cardiomyocyte cell therapy to the subject; and

(b) two doses of tacrolimus and two doses of MPA, a pharmaceutical salt thereof, and/or a proform thereof are administered daily to the subject (bid), optionally about every 12 hours, beginning at the first doses of tacrolimus and MPA, a pharmaceutical salt thereof, and/or a proform thereof and continuously thereafter for the lifetime of the administered cardiomyocyte cell therapy.

54. The method of any of embodiments 1-53, wherein MPA, a pharmaceutical salt thereof, and/or a proform thereof is mycophenolate mofetil (MMF).
55. The method of any of embodiments 1-26, 28, 30-46, and 48-54, wherein:

(i) MPA, a pharmaceutical salt thereof, and/or a proform thereof is mycophenolate mofetil (MMF) and administration of MMF comprises intravenous administration; and

(ii) administration of tacrolimus comprises subcutaneous administration.

56. The method of embodiment 54 or embodiment 55, wherein the total daily dosage of MMF is between about 500 mg and about 3000 mg, between about 1000 mg and about 2500 mg, or between about 1500 mg and about 2000 mg, inclusive of each.
57. The method of any of embodiments 54-56, wherein the total daily dosage of MMF is about 1000 mg, about 1500 mg, about 2000 mg, or about 2500 mg.
58. The method of any of embodiments 1-54, wherein MPA, a pharmaceutical salt thereof, and/or a proform thereof is mycophenolate sodium (MS).
59. The method of embodiment 58, wherein the total daily dosage of MS is between about 360 mg and about 2700 mg, between about 720 mg and about 2160 mg, or between about 720 mg and about 1620 mg, inclusive of each.
60. The method of embodiment 58 or embodiment 59, wherein the total daily dosage of MS is about 360 mg, about 720 mg, about 1080 mg, or about 1440 mg.
61. The method of any of embodiments 1-60, wherein the cardiomyocytes are allogeneic to the subject.
62. The method of any of embodiments 1-61, wherein the cardiomyocytes are primary cardiomyocytes.
63. The method of any of embodiments 1-61, wherein the cardiomyocytes are derived from pluripotent stem cells (PSCs).
64. The method of embodiment 63, wherein the PSCs are induced PSCs (iPSCs).
65. The method of any of embodiments 1-64, wherein administration of the cardiomyocyte cell therapy comprises delivery into a subject's heart tissue, optionally by intravenous injection, intraarterial injection, intracoronary injection, intramuscular injection, intraperitoneal injection, intramyocardial injection, trans-endocardial injection, trans-epicardial injection, and/or infusion.
66. The method of any of embodiments 1-65, wherein the cardiomyocyte cell therapy comprises between about 5×10⁸ and 1×10¹⁰ cardiomyocytes, inclusive of each.
67. The method of any of embodiments 1-66, wherein the cardiomyocyte cell therapy comprises between about 1×10⁹ and about 5×10⁹ cardiomyocytes, inclusive of each.
68. The method of any of embodiments 1-67, wherein the cardiomyocyte cell therapy comprises a pharmaceutically acceptable carrier.
69. The method of any of embodiments 1-68, wherein the subject has a heart disease or condition.
70. The method of embodiment 69, wherein the heart disease or condition is pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, other cardiomyopathy, myocarditis, myocardial infarction, myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, or cardiovascular disease.
71. The method of embodiment 69 or embodiment 70, wherein the heart disease or condition is myocardial infarction (MI).
72. The method of any of embodiments 1-71, wherein the subject is a candidate for a Left Ventricular Assist Device (LVAD).
73. The method of any of embodiments 1-72, wherein the subject has a LVAD at the time of administration of the cardiomyocyte cell therapy and/or at the time of administration of tacrolimus and MPA, a pharmaceutical salt thereof, and/or a proform thereof.
74. The method of any of embodiments 1-73, wherein the subject is a human.
75. The method of any of embodiments 1-74, wherein the cardiomyocytes are human.
76. Use of (i) tacrolimus and (ii) mycophenolic acid (MPA), a pharmaceutical salt thereof, and/or a proform thereof for attenuating or preventing rejection of a cardiomyocyte cell therapy in a subject who has received, is receiving, and/or is to receive a cardiomyocyte cell therapy.
77. Use of (i) tacrolimus and (ii) mycophenolic acid (MPA), a pharmaceutical salt thereof, and/or a proform thereof in the manufacture of a medicament for attenuating or preventing rejection of a cardiomyocyte cell therapy in a subject who has received, is receiving, and/or is to receive a cardiomyocyte cell therapy.

### VI. EXAMPLES

The following example is included for illustrative purposes only and is not intended to limit the scope of the invention.

### Example 1: Use of an Immunosuppressive Therapy in a Non-Human Primate Model of Allogeneic Cardiomyocyte Cell Therapy

The ability of an immunosuppressive therapy of tacrolimus and mycophenolate mofetil (MMF) to prevent rejection following delivery of an allogeneic induced pluripotent stem cell (iPSC)-derived cardiomyocyte cell therapy composition was assessed in a non-human primate model of acute myocardial infarction (MI).

Allogeneic iPSCs isolated from rhesus macaque (Macaca mulatta, riPSCs) were differentiated into a highly enriched population of cardiomyocytes (riPSC-CMs). Briefly, riPSCs were thawed and cultured for approximately 25 days, with regular passaging and media exchange, for differentiation into cardiomyocytes (riPSC-CMs). The differentiated riPSC-CMs were then harvested and cryopreserved for future use.

200×10⁶ riPSC-CMs were thawed and resuspended in a pharmaceutically acceptable media, and delivered as a cell therapy composition into a MHC-mismatched rhesus recipient at the site of the previous myocardial infarction.

Beginning five days prior to delivery of the cell therapy composition and continuing for 16 weeks after delivery, the rhesus recipient was treated daily with tacrolimus and MMF. Tacrolimus was provided subcutaneously twice per day at a dose yielding a blood trough level of approximately 5-10 ng/mL. MMF was provided orally twice per day, for a total dose of 250 mg MMF per day. If a subject cannot tolerate oral dosing of MMF, MMF can be alternatively provided by intravenous administration. In some cases, if a subject cannot tolerate oral MMF, the subject is administered intravenous MMF until the subject can tolerate oral MMF, at which point the subject is switched from intravenous to oral MMF Immune rejection of the riPSC-CMs 16 weeks after delivery was determined by histopathological analysis.

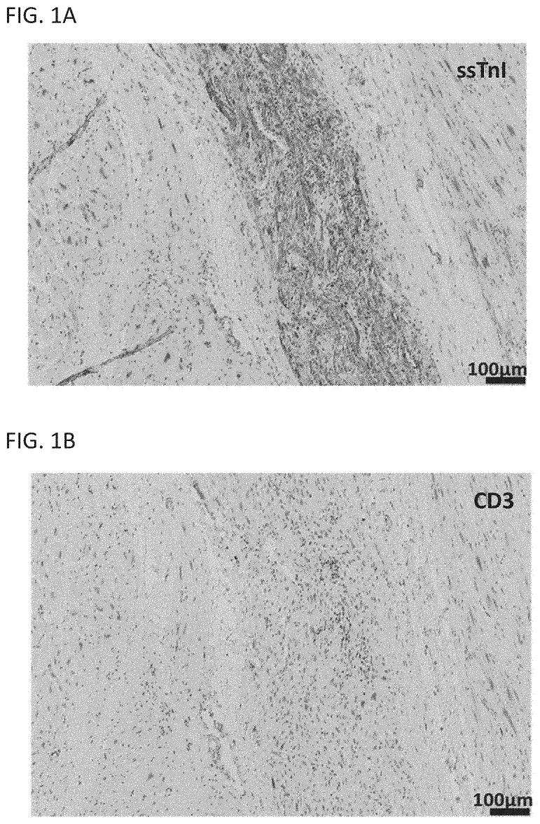

Minimal rejection of the riPSC-CMs was observed at the site of the myocardial infarction 16 weeks following delivery of the cells, as shown by straining for ssTnI, a marker of the riPSC-CMs (FIG. 1A) and CD3, a marker of T cells (FIG. 1B).

These results are consistent with a finding that an immunosuppressive therapy of tacrolimus and MMF allows allogeneic cardiomyocytes delivered as a cell therapy composition to persist at the site of engraftment.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method of attenuating or preventing rejection of a cardiomyocyte cell therapy in a subject, the method comprising administering to a subject (i) tacrolimus and (ii) mycophenolic acid (MPA), a pharmaceutical salt thereof, or a proform thereof, each prior to, concurrent with, or subsequent to administration of a cardiomyocyte cell therapy to the subject.

2. A method of attenuating or preventing rejection of a cardiomyocyte cell therapy in a subject, the method comprising administering to a subject a cardiomyocyte cell therapy prior to, concurrent with, or subsequent to administration of (i) tacrolimus and (ii) mycophenolic acid (MPA), a pharmaceutical salt thereof, or a proform thereof to the subject.

3. A method of attenuating or preventing rejection of a cardiomyocyte cell therapy in a subject, the method comprising:
(1) administering a cardiomyocyte cell therapy to a subject; and
(2) administering (i) tacrolimus and (ii) mycophenolic acid (MPA), a pharmaceutical salt thereof, or a proform thereof to the subject, wherein administration of (i) tacrolimus and (ii) MPA, a pharmaceutical salt thereof, or a proform thereof is prior to, concurrent with, or subsequent to administration of the cardiomyocyte cell therapy to the subject.

4. The method of claim 3, wherein the cardiomyocyte cell therapy is a pharmaceutical composition comprising cardiomyocytes and a pharmaceutically acceptable carrier.

5. The method of claim 3, wherein the cardiomyocyte cell therapy is administered as a suspension of cardiomyocytes or as an engineered tissue graft comprising cardiomyocytes and a matrix.

6. (canceled)

7. The method of claim 3, wherein administration of tacrolimus comprises administration of at least one dose of tacrolimus prior to, concurrent with, or subsequent to administration of the cardiomyocyte cell therapy to the subject.

8-9. (canceled)

10. The method of claim 3, wherein a first dose of tacrolimus is administered to the subject about 5 days prior to administration of the cardiomyocyte cell therapy to the subject.

11-12. (canceled)

13. The method of claim 3, wherein administration of tacrolimus comprises administration of at least one dose of tacrolimus subsequent to administration of the cardiomyocyte cell therapy to the subject.

14-15. (canceled)

16. The method of claim 3, wherein the total daily dosage of tacrolimus administered to the subject yields a blood trough level of between about 1 ng/mL and about 30 ng/mL, inclusive of each.

17-18. (canceled)

19. The method of claim 3, wherein tacrolimus is administered at least once daily.

20. The method of claim 3, wherein the total daily dosage of tacrolimus is provided as a single dose per day, or is divided between 2 doses, 3 doses, or 4 doses per day.

21-22. (canceled)

23. The method of claim 3, wherein tacrolimus is administered daily for up to about 60 months subsequent to administration of the cardiomyocyte cell therapy to the subject.

24. The method of claim 3, wherein tacrolimus is administered daily for the lifetime of the cardiomyocyte cell therapy in the subject.

25. The method of claim 3, wherein:
(a) a first dose of tacrolimus is administered to the subject about 5 days prior to administration of the cardiomyocyte cell therapy to the subject; and
(b) two doses of tacrolimus are administered daily to the subject (bid) beginning at the first dose of tacrolimus and continuously thereafter for the lifetime of the administered cardiomyocyte cell therapy.

26. The method of claim 3, wherein administration of tacrolimus comprises oral, subcutaneous, or intravenous administration.

27-29. (canceled)

30. The method of claim 3, wherein administration of MPA, a pharmaceutical salt thereof, or a proform thereof comprises administration of at least one dose of MPA, a pharmaceutical salt thereof, or a proform thereof prior to, concurrent with, or subsequent to administration of the cardiomyocyte cell therapy to the subject.

31-32. (canceled)

33. The method of claim 3, wherein a first dose of MPA, a pharmaceutical salt thereof, or a proform thereof is administered to the subject about 5 days prior to administration of the cardiomyocyte cell therapy to the subject.

34-35. (canceled)

36. The method of claim 3, wherein administration of MPA, a pharmaceutical salt thereof, or a proform thereof comprises administration of at least one dose of MPA, a pharmaceutical salt thereof, or a proform thereof subsequent to administration of the cardiomyocyte cell therapy to the subject.

37-38. (canceled)

39. The method of claim 3, wherein MPA, a pharmaceutical salt thereof, or a proform thereof is administered at least once daily.

40. The method of claim 3, wherein the total daily dosage of MPA, a pharmaceutical salt thereof, or a proform thereof is provided as a single dose per day, or is divided between two doses, three doses, or four doses per day.

41-42. (canceled)

43. The method of claim 3, wherein MPA, a pharmaceutical salt thereof, or a proform thereof is administered daily for up to about 60 months subsequent to administration of the cardiomyocyte cell therapy to the subject.

44. The method of claim 3, wherein MPA, a pharmaceutical salt thereof, or a proform thereof is administered daily for the lifetime of the administered cardiomyocyte cell therapy.

45. The method of claim 3, wherein:
(a) a first dose of MPA, a pharmaceutical salt thereof, or a proform thereof is administered to the subject about 5 days prior to administration of the cardiomyocyte cell therapy to the subject; and
(b) two doses of MPA, a pharmaceutical salt thereof, or a proform thereof are administered daily to the subject (bid), optionally about every 12 hours, beginning at the first dose and continuously thereafter for the lifetime of the administered cardiomyocyte cell therapy.

46. The method of claim 3, wherein administration of MPA, a pharmaceutical salt thereof, or a proform thereof comprises oral, subcutaneous, or intravenous administration.

47-49. (canceled)

50. The method of claim 3, wherein:
(i) a first dose of tacrolimus and a first dose of MPA, a pharmaceutical salt thereof, or a proform thereof are each administered to the subject on the same day;
(ii) tacrolimus and MPA, a pharmaceutical salt thereof, or a proform thereof are each administered to the subject daily; or
(iii) the total daily dosage of tacrolimus is divided between two doses per day and the total daily dosage of MPA, a pharmaceutical salt thereof, or a proform thereof is divided between two doses per day.

51-52. (canceled)

53. The method of claim 3, wherein:
(a) a first dose of tacrolimus and a first dose of MPA, a pharmaceutical salt thereof, or a proform thereof are each administered to the subject about 5 days prior to administration of the cardiomyocyte cell therapy to the subject; and
(b) two doses of tacrolimus and two doses of MPA, a pharmaceutical salt thereof, or a proform thereof are administered daily to the subject (bid) beginning at the first doses of tacrolimus and MPA, a pharmaceutical salt thereof, or a proform thereof and continuously thereafter for the lifetime of the administered cardiomyocyte cell therapy.

54. The method of claim 3, wherein MPA, a pharmaceutical salt thereof, and/or a proform thereof is mycophenolate mofetil (MMF).

55. (canceled)

56. The method of claim 54, wherein the total daily dosage of MMF is between about 500 mg and about 3000 mg.

57. (canceled)

58. The method of claim 3, wherein MPA, a pharmaceutical salt thereof, or a proform thereof is mycophenolate sodium (MS).

59-60. (canceled)

61. The method of claim 3, wherein the cardiomyocytes are allogeneic to the subject.

62. The method of claim 3, wherein the cardiomyocytes are primary cardiomyocytes.

63. The method of claim 3, wherein the cardiomyocytes are derived from pluripotent stem cells (PSCs).

64. (canceled)

65. The method of claim 3, wherein administration of the cardiomyocyte cell therapy comprises delivery into a subject's heart tissue.

66. The method of claim 3, wherein the cardiomyocyte cell therapy comprises between about 5×10⁸ and 1×10¹⁰ cardiomyocytes, inclusive of each.

67-68. (canceled)

69. The method of claim 3, wherein the subject has a heart disease or condition.

70. (canceled)

71. The method of claim 69, wherein the heart disease or condition is myocardial infarction (MI).

72. The method of claim 3, wherein the subject (i) is a candidate for a Left Ventricular Assist Device (LVAD); (ii) has a LVAD at the time of administration of the cardiomyocyte cell therapy; or (iii) has a LVAD at the time of administration of tacrolimus and MPA, a pharmaceutical salt thereof, or a proform thereof.

73-77. (canceled)

78. The method of claim 1, comprising administering to the subject (i) tacrolimus and (ii) mycophenolic acid (MPA), a pharmaceutical salt thereof, or a proform thereof, each prior to administration of the cardiomyocyte cell therapy to the subject.

79. The method of claim 2, comprising administering the cardiomyocyte cell therapy to the subject subsequent to administration of (i) tacrolimus and (ii) mycophenolic acid (MPA), a pharmaceutical salt thereof, or a proform thereof to the subject.

80. The method of claim 3, wherein administration of (i) tacrolimus and (ii) MPA, a pharmaceutical salt thereof, or a proform thereof to the subject is prior to administration of the cardiomyocyte cell therapy to the subject.
